SEQUENCE LISTING (4 sequences)

SEQ ID NO: 1 - RNA, 19 nt, synthetic construct
  source 1..19: mol_type = other RNA
caaggcagctctacactca

SEQ ID NO: 2 - RNA, 19 nt, synthetic construct
  source 1..19: mol_type = other RNA
gagacgggctgctccatga

SEQ ID NO: 3 - RNA, 19 nt, synthetic construct
  source 1..19: mol_type = other RNA
gctcctccatggcgagttt

SEQ ID NO: 4 - RNA, 19 nt, synthetic construct
  source 1..19: mol_type = other RNA
ccactgccctcacctgtct

(19) United States
(12) Patent Application Publication
(10) Pub. No.: US 2024/0401058 A1
(43) Pub. Date: Dec. 5, 2024

(54) METHOD OF REGULATING PULMONARY ARTERY VASCULAR REMODELING

(71) Applicant: THE TRUSTEES OF INDIANA UNIVERSITY, Bloomington, IN (US)

(72) Inventors: Margaret Arlene SCHWARZ, Bristol, IN (US); Arambage Dushani RANASIGNHE, South Bend, IN (US)

(21) Appl. No.: 18/699,774

(22) PCT Filed: Oct. 19, 2022
(86) PCT No.: PCT/US2022/078376

§ 371 Date: Apr. 9, 2024

Related U.S. Application Data
(60) Provisional application No. 63/257,269, filed on Oct. 19, 2021.

(51) Int. Cl. C12N 15/113; A61K 31/4409

(52) U.S. Cl. CPC C12N 15/1137; A61K 31/4409; C12N 2310/14

(57) ABSTRACT

Disclosed herein are compositions and methods for treating pulmonary arterial hypertension. Compositions comprising an inhibitor of sphingosine kinase 2 can be used to reduce pulmonary artery vascular remodeling in a patient with pulmonary arterial hypertension.

### CROSS REFERENCE TO RELATED APPLICATIONS

This application claims priority to the following: U.S. Provisional Patent Application No. 63/257,269 filed on Oct. 19, 2021, the disclosure of which is expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 5 kilobytes xml file named “29920-372720.xml” created on Oct. 16, 2022.

### BACKGROUND

Pulmonary arterial hypertension (PAH) is a progressive, incurable and devastating cardiopulmonary vascular disease with a 3-year survival rate <60% and sex-related epidemiological and pathophysiological differences. PAH is defined by obliteration of proximal pulmonary vessels, loss of distal microvasculature, and right sided heart failure. Risk factors and sources of vascular injury in PAH vascular remodeling are diverse including host factors such as genetic mutations, sex hormones and age or acquired and environmental factors like hypoxia, somatic mutations, dysfunctional immune/inflammatory response, pathogens, and drugs. Accumulation of enough “hits” to the pulmonary vasculature, can lead to uncontrolled pulmonary arterial smooth muscle cell (PASMC) proliferation that mirrors cancer cell phenotypes with aberrant cellular signaling and uncontrolled proliferation. Being multifactorial in nature, the etiology of PAH is diverse with familial or heritable PAH being associated with genetic mutations while the most common form, idiopathic PAH (IPAH) lacks information regarding causal or genetic defects. The cardinal pathophysiological hallmark of PAH is progressive, obliterative vascular remodeling that gives rise to increased pulmonary vascular resistance (PVR) and right ventricular overload. Unfortunately, current PAH therapy offers only symptomatic relief, targeting vasoconstriction and heart failure but not pathologic arterial wall thickening and remodeling. Thus, fundamental gaps remain in our understanding of the underlying mechanism mediating the pathophysiological influences in PAH that modulate pulmonary vascular remodeling.

Recently there is an emerging recognition of the fundamental role that acquired abnormalities, functioning as epigenetic modifications, have in contributing to the establishment and progression of PAH. While epigenetic factors drive aberrant gene transcription in PAH endothelial and smooth muscle cells, thus contributing to vascular remodeling, factors that mediate this process in PAH are poorly understood. However, the recognition of epigenetic factor's role in PAH is important as this discovery could facilitate the development of novel therapeutic approaches, diagnostic modalities, and biomarkers. Epigenetics has an emerging role in the development of IPAH as modified histone proteins are reported to play a vital role in PAH pathogenesis. A recent study showed altered histone deacetylation to be associated with PAH by impairing redox signaling and creating a proliferative, apoptosis-resistant pulmonary arterial smooth muscle cell (PASMC). In contrast to the detailed examined pattern of DNA methylation and histone methylation, the data of the distribution of the acetylated histone proteins in PAH and vascular biology are still incomplete.

Acetylated H3K9 (Ac-H3K9) is a significant marker of transcription and transcribed genes, and a high intensity of H3K9 acetylation has been detected around the transcription start site of downstream genes including promoters and enhancers. Furthermore, Ac-H3K9 is known to be involved in cell hyper-proliferation and inflammation which are key pathologic characteristics seen in PAH settings. Accordingly, as disclosed herein the role of H3K9 (Ac-H3K9) in PAH pathogenesis was investigated.

The bioactive sphingolipid metabolite, sphingosine 1-phosphate (S1P) is known to be involved in differential histone acetylation in different cell types of disease pathogenesis including cancer cells that also display uncontrolled cell proliferation similar to PASMC in PAH. Moreover, previous studies demonstrate that as a predominant lipid constituent of all cellular membranes, sphingolipid homeostasis mediates the adaption of cells to their external environment with S1P demonstrating an important role in occlusive arteriopathy via PASMC proliferation. Although, S1P has been identified as a formidable nuclear epigenetic modulator in the other disease processes, the epigenetic role of S1P in PAH has not been studied. Generation of S1P is catalyzed by two distinct sphingosine kinase (SPHK) isoenzymes SPHK1 and SPHK2 that possess identical kinetic domains but distinct kinetic properties, distribution, regulation and functions. SPHK1 mainly localizes to the cytosol while SPHK2 is located in cytoplasm and nucleus, with nuclear localization and export signals found on its four splice variants (SPHK2-a,b,c,d). Nuclear SPHK2 phosphorylates sphingosine to S1P. Nuclear S1P unleashes transcription co-repressor complexes to activate latent gene transcription by acting as an endogenous inhibitor of histone deacetylases (HDACs).

Despite a key role for SPHK2 in the epigenetic mediated disease progression in other disease process, its role in PAH has been overlooked. This is in part due to prior PAH studies that explored SPHK2's role through use of an N-terminal antibody that recognized only one of the four SPHK2 isoforms (only SPHK2b) thus omitting the expression of the other three isoforms. As a result, SPHK2's role in PAH has been marginalized.

As disclosed herein applicants have discovered there is a 20-fold increase in SPHK2 expression in human PAH lung tissue unveiling its potential role as a novel epigenetic contributor to PAH vascular remodeling. Furthermore, by utilizing a hypoxia mouse model of PAH, human IPAH lung tissues and in vitro human PASMC, applicants have discovered that the active chromatin mark H3K9 acetylation and SPHK2/S1P axis have a role in PAH vascular remodeling.

### SUMMARY

Our in vivo studies in hypoxia experimental mouse models determined that SPHK2 ablation has a protective role against PAH. SPHK2 expression is significantly elevated in IPAH lung tissue derived from transplanted IPAH patients as compared to failed donor lungs. A known epigenetically modified SPHK2 target, Ac-H3K9 and its downstream transcription machinery of known vascular remodeling gene Kruppel-like factor (KLF4), were significantly increased. Importantly, a key sex difference was noted as tissue from female patients demonstrated a greater significant increase in Ac-H3K9 and its downstream target KLF4 as compared to their male counterparts. In addition, we determined that SPHK2 phosphorylation, nuclear expression, and mediation of gene transcription is activated by a known pro-inflammatory mediator of vascular growth and remodeling process, Endothelial Monocyte Activating Polypeptide II (EMAPII).

Taken together, this data suggests that histone H3K9 acetylation acts as an EMAPII/SPHK2/S1P-sensitive active epigenetic upstream signaling pathway that promotes PASMCs proliferation and PAH vascular remodeling. Our findings are an important discovery for PAH, as the epigenetic modulator SPHK2 represents a new therapeutic target that could provide opportunities for the development of novel PAH therapies.

The targeting of factors that drive pulmonary vascular remodeling in patients with fatal pulmonary hypertension would represent a novel therapeutic approach to the treatment of pulmonary hypertension. This is vital as to date there are no existing therapies that target pulmonary vascular remodeling. As disclosed herein activated nuclear SPHK2 promotes distal pulmonary vascular remodeling through epigenetic modifications that reawaken genes that are associated with progression of Pulmonary hypertension and thus contribute to PAH vascular obliteration. Accordingly, one embodiment of the present invention is directed to the use of SphK2 inhibitors to inhibit PAH associated obliteration of distal pulmonary vessels as a treatment of PAH. Through inhibition of pSPHK2 epigenetic mediated remodeling of pulmonary vasculature, progression and reversal of pulmonary vascular remodeling will become a novel therapeutic approach to the treatment of pulmonary hypertension.

In accordance with one embodiment a method of reducing pulmonary artery vascular remodeling in a patient diagnosed with pulmonary arterial hypertension is provided. In one embodiment the method comprises administering a pharmaceutical composition that comprises a agent that interferes or prevents acetylation of H3K9. In one embodiment the pharmaceutical composition comprises an inhibitor of sphingosine kinase 2. Accordingly, in one embodiment a method of reducing pulmonary artery vascular remodeling in a patient diagnosed with pulmonary arterial hypertension comprises administering an inhibitor of sphingosine kinase 2. In accordance with the present disclosure an inhibitor of sphingosine kinase 2 can be any moiety that decreases the activity of sphingosine kinase 2 in the cells of a patient. This can include interfering with the expression of sphingosine kinase 2 (e.g., use of interfering RNAs) or impacting the stability or destruction of the encoded protein or interfering with the functional activity of the protein (e.g., use of small molecule inhibitors or antibodies). In accordance with one embodiment an interfering RNA selected from the group consisting of SEQ ID NO: 1-4 is administered a patient to decrease the activity of sphingosine kinase 2 in the cells of a patient.

In one embodiment the inhibitor of sphingosine kinase 2 is an interfering RNA that targets sphingosine kinase 2. In another embodiment the inhibitor comprises a compound of the general structure:

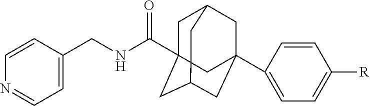

where R is halo.

In one embodiment a method of treating pulmonary arterial hypertension is provided, wherein the method comprises decreasing H3K9 acetylation in the cells of a patient diagnosed with pulmonary arterial hypertension. In one embodiment the method of decreasing H3K9 acetylation comprises administration of a pharmaceutical composition comprising an agent that
i) decreases sphingosine kinase 2 activity; or
ii) decreases Kruppel-like factor (KLF4) activity; or
iii) a combination of i) and ii).
   In one embodiment a method of treating pulmonary arterial hypertension is provided wherein the method comprises administering an inhibitor of sphingosine kinase 2 to said patient. In one embodiment the inhibitor is an interfering RNA that targets sphingosine kinase 2. In one embodiment the inhibitor comprises a compound having the general structure:

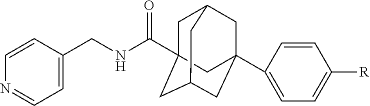

where R is halo.

### BRIEF DESCRIPTION OF THE DRAWINGS

FIGS. 1A-1E: H3K9 acetylation and SPHK2 expression show a potential correlation in IPAH patients' lungs. FIG. 1A is a representative immunoblot probed for Ac-H3K9 and total H3 in protein lysates of human IPAH or failed donor lung (FDL) specimens and the ponceau stain of the immunoblot. FIG. 1B is a graph presenting the quantitation of Ac-H3K9/Total H3. A representative immunoblot probed for SPHK2 and Vinculin (loading control) in protein lysates of human IPAH or FDL specimens is shown in FIG. 1C. FIG. 1D is a graph presenting the quantitation of SPHK2/Vinculin; and SPHK2 expression levels normalized against 1BS rRNA is shown in FIG. 1E. Following two-way ANOVA, Sfdak's multiple comparisons test, *p<0.05, **p<0.01. Results are shown as means±SEM. n=6.

FIGS. 2A-2E: SPHK2 ablation confers protection against PAH and hyperacetylation of H3K9 in hypoxia-induced experimental PAH mouse model. SPHK2 KO or wild type control mice (C57BL/6NJ) were subjected to 3 weeks of hypoxia (10% O₂) or normoxia (room air). FIG. 2A presents data measuring pulmonary vascular resistance (the maximum velocity of tricuspid regurgitation/the velocity time integral of the right ventricular outflow tract, TRmax velocity/VTIRVOT) n=8-10. FIG. 2B presents data measuring RV hypertrophy/Fulton Index (the weight ratio of the right ventricle divided by the sum of left ventricle and septum, RV/(LV+S)) n=8-10. FIG. 2C presents data quantifying elastin-stained distal pulmonary vessels and wall thickness of distal pulmonary vessels in elastin-stained lung tissue sections (wall thickness (%)=(2× medial wall thickness/external diameter)×100) n=3 randomly selected mice per each group and n=15 images of distal pulmonary vessels, scale bar is 10 μm. A representative immunoblot probed for Ac-H3K9 and total H3 in whole tissue lysates n=5 is shown in FIG. 2D. FIG. 2E presents data from quantitation of immunoblots probed for Ac-H3K9 and total H3 in whole tissue lysates n=5 Ac-H3K9/Total H3. In summary, SPHK2 ablation inhibits hyperacetylation of H3K9 resulting in protection against PAH. Created with BioRender.com. Following one-way ANOVA, Tukey's multiple comparisons test, *p<0.05, **p<0.01, ***p<0.001. Results are shown as means±SEM.

FIGS. 3A-3F: Pulmonary expression of KLF4 is upregulated in IPAH patients and hypoxia-induced experimental PAH mouse model. A representative immunoblot probed for KLF4, SOX2 and Vinculin in protein lysates of human IPAH or failed donor lung (FDL) specimens is shown in FIG. 3A. Immunoblots were probed for KLF4, SOX2 and Vinculin in protein lysates of human IPAH or failed donor lung (FDL) specimens and the quantitation of KLF4 and SOX 2 against Vinculin is shown in FIG. 3B. FIG. 3C provides a graph of KLF4 and SOX2 expression levels normalized against 1BS rRNA utilizing cDNA synthesized from RNA extracts of human IPAH or FDL specimens. A representative immunoblot probed for KLF4 and Tubulin (loading control) in protein lysates from SPHK2 KO or wild type control mice that were subjected to 3 wks of hypoxia (10% O₂) or normoxia is shown in FIG. 3D. Quantification of KLF4/Tubulin was determined as shown in FIG. 3E. KLF4 expression levels normalized against Hprt1 utilizing cDNA synthesized from RNA extracts of SPHK2 KO or wild type control mice that were exposed to 3 weeks of hypoxia (10% O₂) or normoxia (room air) is shown in FIG. 3F. Following two-way ANOVA, Sfdak's multiple comparisons test or one-way ANOVA, Tukey's multiple comparisons test, *p<0.05, **p<0.01. Results are shown as means±SEM. n=5 or 6.

FIG. 4A-4F: EMAP II has the potential to be a mediator of SPHK2 signaling axis in pulmonary artery smooth muscle cells. The proinflammatory cytokine, EMAP II is upregulated in PAH pathogenesis causing the activation of SPHK2 signaling axis to induce hyperacetylation of pluripotency factor KLF4 that will in return induces PASMC proliferation leading to PAH. Evidence supporting this pathways is provides as follows: Immunoblots were probed for AIMP1 (precursor form of EMAP II) and Tubulin (loading control) in protein lysates from SPHK2 KO or wild type control mice that were subjected to 3 weeks of hypoxia (10% O₂) or normoxia n=5 and quantitation of AIMP1/Tubulin is shown in FIG. 4A. Immunoblots were probed for AIMP1 and Tubulin in protein lysates of human IPAH or failed donor lung (FDL) specimens n=6 and a representative immunoblot is shown in FIG. 4B. Quantitation of AIMP1 against Vinculin is shown in FIG. 4C. A representative immunoblot probed for AIMP1 and Vinculin in protein lysates of human IPAH or failed donor lung (FDL) specimens n=6 is shown in FIG. 4D. Immunoblots were probed for pSPHK2, SPHK2, tubulin and lamin B in cytoplasmic and nuclear fractions of hPASMC following EMAP II treatment for 0, 2, 4, 12 and 24 hours n=3 and quantification of nuclear pSPHK2/lamin B is shown in FIG. 4E. FIG. 4F is a representative immunoblot probed for pSPHK2, SPHK2, tubulin and lamin B in cytoplasmic and nuclear fractions of hPASMC following EMAP II treatment for 0, 2, 4, 12 and 24 hours n=3). Following two-way ANOVA, Sfdak's multiple comparisons test or one-way ANOVA, Tukey's multiple comparisons test, *p<0.05, **p<0.01. Results are shown as means±SEM.

FIGS. 5A-5G: EMAP II promotes activation and nuclear translocation of SPHK2 that generates nuclear S1P and inhibits HDAC1. Immunocytochemistry images of pSPHK2, actin (cytoplasmic marker) and DAPI (nuclear) coimmunostaining in EMAP II treated (2 hr) or vehicle treated fixed hPASMC, were quantified by fluorescence intensity of pSPHK2 and the results are shown in FIG. 5A. A representative immunoblot probed for pSPHK2 and lamin B in nuclear fractions of hPASMC following EMAP II treatment (150 minutes) with or without SPHK2 inhibitor is shown in FIG. 5B. Immunoblots were probed for pSPHK2 and lamin B in nuclear fractions of hPASMC following EMAP II treatment (150 minutes) with or without SPHK2 and quantification of nuclear pSPHK2/lamin B is shown in FIG. 5C. FIG. 5D shows the results of ELISA-nuclear C18-S1P levels, normalized against 1 μg of nuclear proteins in the nuclear fractions of hPASMC cells, following EMAP II treatment for 0, 2, 4, 12 and 24 hours. FIG. 5E shows the results of ELISA-nuclear C18-S1P levels, normalized against 1 μg of nuclear proteins in the nuclear fractions of hPASMC cells, following EMAP II for 15 or 150 minutes with or without SPHK2 inhibitor. FIG. 5F shows HDAC activity normalized against 1 μg of nuclear proteins in the nuclear fractions of hPASMC cells following EMAP II for 150 minutes with or without SPHK2 inhibitor. FIG. 5G shows cell proliferation rate in hPASMC cells pretreated with SPHK2 or SPHK1 inhibitors or vehicle following EMAP II treatment for 24 hours. Following unpaired t-test or one-way ANOVA, Tukey's multiple comparisons test, *p<0.05, **p<0.01, ***p<0.001, ****p<0.0001. Results are shown as means±SEM and n=3.

FIGS. 6A-6G: EMAP II mediated SPHK2 signaling promotes hyperacetylation of H3K9 in hPASMC. A representative immunoblot probed for Ac-H3K9, total H3 or tubulin in hPASMC cells following EMAP II treatment for 0, 1, 2, 4, 6, 12 and 24 hours is shown in FIG. 6A. Immunoblots were probed for Ac-H3K9, total H3 or tubulin in hPASMC cells following EMAP II treatment for 0, 1, 2, 4, 6, 12 and 24 hours and quantitation of Ac-H3K9 expression levels were normalized against total H3 (see FIG. 6B). A representative immunoblot probed for HDAC1 and tubulin in hPASMC cells following EMAP II treatment for 0, 1. 2, 4, 6, 12 and 24 hours is shown in FIG. 6C. Immunoblots were probed for HDAC1 and tubulin in hPASMC cells following EMAP II treatment for 0, 1. 2, 4, 6, 12 and 24 hours and quantitation of HDAC1/Tubulin was conducted with the results shown in FIG. 6D. A representative immunoblot probed for Ac-H3K9, total H3, SPHK2 and tubulin in whole cell lysates of hPASMC cells following siRNA mediated SPHK2 silencing and EMAP II/EMAP II neutralizing antibody treatment for 4 hours is shown in FIG. 6E. Immunoblots were probed for Ac-H3K9, total H3, SPHK2 and tubulin in whole cell lysates of hPASMC cells following siRNA mediated SPHK2 silencing and EMAP II/EMAP II neutralizing antibody treatment for 4 hours and quantitation of Ac-H3K9/total H3 (FIG. 6F) and quantification of SPHK2/tubulin (FIG. 6G) were conducted. one-way ANOVA, Tukey's multiple comparisons test, *p<0.05, **p<0.01, p<0.001, ****p<0.0001. Results are shown as means±SEM and n=3.

FIGS. 7A-7D: EMAP II mediated SPHK2 signaling promotes hyperacetylation of H3K9 in KLF4 leading to increased expression of KLF4 in hPASMC. FIG. 7A is a graph demonstrating the number of peaks of Ac-H3K9 normalized to IgG in with or without SPHK2 inhibitor and EMAP II treated (2-3 hours) hPASMC. Representative immunoblots probed for KLF4, SPHK2 and tubulin in whole cell lysates of hPASMC cells following siRNA mediated SPHK2 silencing and EMAP II/EMAP II neutralizing antibody treatment for 6-8 hours are shown in FIG. 7B. Immunoblots were probed for KLF4, SPHK2 and tubulin in whole cell lysates of hPASMC cells following siRNA mediated SPHK2 silencing and EMAP II/EMAP II neutralizing antibody treatment for 6-8 hours, and quantitation of KLF4/tubulin (FIG. 7C) and KLF4 (FIG. 7D) expression levels normalized against 1BS rRNA in hPASMC cells following siRNA mediated SPHK2 silencing and EMAP II treatment for 6 hours. In summary, the data indicates that SPHK2/S1P signaling axis along with EMAP II promotes PASMC proliferation through HDAC expression and activity inhibition, which in turn results in hyperacetylated H3K9 regions in KLF4 resulting in overexpression of KLF4 that promotes PASMC proliferation in PAH. Created with BioRender.com. one-way ANOVA, Tukey's multiple comparisons test, *p<0.05, **p<0.01, ***p<0.001. Results are shown as means±SEM and n=3.

FIGS. 8A-8G: EMAP II has the potential to be a key pathogenic mediator in PH through modulating epigenetic equilibrium via histone H3K9 acetylation uniquely in vascular SMCs. FIG. 8A: Representative immunoblot probed for AIMP1 (precursor form of EMAP II) and Tubulin in protein lysates of human iPAH or FDL, n=20/group and, FIG. 8B provides quantitation of AIMP1 (AIMP1/Tubulin) in protein lysates of human iPAH or FDL, n=20/group. FIG. 8C: Representative immunoblot probed for Ac-H3K9, total H3 or tubulin in hPASMCs following EMAP II treatment for 0, 1, 2, 4 and 6 hours and FIG. 8D provides quantitation of Ac-H3K9 expression levels normalized against total H3 in hPASMCs, n=4. FIG. 8E: Representative immunoblot probed for Ac-H3K9, total H3 or tubulin in hPMVECs following EMAP II treatment for 0, 1, 2, 4 and 6 hours and FIG. 8F provides quantitation of Ac-H3K9 expression levels normalized against total H3 in hPMVECs, n=3. Following unpaired t-test or Kolmogorov-Smirnov non-parametric testing, *p<0.05, **p<0.01. Results are shown as means±SEM. FIG. 8G: Immunohistochemical analysis and previous studies determined that AIMP1 expression was elevated in microvascular regions within the thickened remodeled vessels of iPAH lungs

FIGS. 9A-9F: EMAP II promotes nuclear activation of SPHK2 that in turn generates nuclear lipid, S1P in vascular SMCs. FIG. 9A: Representative immunoblot probed for pSPHK2, SPHK2, tubulin and lamin B in cytoplasmic and nuclear fractions of hPASMCs following EMAP II treatment for 0, 2 and 4 hours, and FIG. 9B provides quantification of nuclear pSPHK2/lamin B, n=3/group. Representative immunocytochemistry images of pSPHK2, actin, cytoplasmic marker) and DAPI (nuclear) were coimmunostained in EMAP II treated (2 hr) or vehicle treated fixed hPASMCs, scale bar is 20 μm. FIG. 9C: Representative immunoblot probed for pSPHK2 and lamin B in nuclear fractions of hPASMCs following EMAP II treatment (150 minutes) with or without SPHK2 inhibitor FIG. 9D provides quantification of nuclear pSPHK2/lamin B. FIG. 9E: ELISA-nuclear C18-S1P levels normalized against 1 μg of nuclear proteins in the nuclear fractions of hPASMCs following EMAP II treatment for 0, 2, 4, 12 and 24 hours. FIG. 9F: ELISA-nuclear C18-S1P levels normalized against 1 μg of nuclear proteins in the nuclear fractions of hPASMCs following EMAP II for 15 or 150 minutes with or without SPHK2 inhibitor. Following Kruskal-Wallis or Kolmogorov-Smirnov non-parametric test, *p<0.05, **p<0.01. Results are shown as means±SEM and n=3 or 4/group.

FIGS. 10A-10E: EMAP II mediated SPHK2 signaling promotes global hyperacetylation of histone H3K9 in vascular SMCs. FIG. 10A: Representative immunoblot probed for Ac-H3K9, total H3, SPHK2 and tubulin in whole cell lysates of hPASMCs following siRNA mediated SPHK2 silencing and post-transfection EMAP II treatment for 4 hours and FIG. 10B provides quantitation of Ac-H3K9/total H3 and FIG. 10C provides quantification of SPHK2/tubulin. FIG. 10D: Number of peaks of Ac-H3K9 normalized to IgG in with or without SPHK2 inhibitor and EMAP II treated (2-3 hours) hPASMCs. FIG. 10E: Cell proliferation rate in hPASMCs treated with vehicle or EMAP II following SPHK2 inhibitor treatment for 24 hours. Following Kruskal-Wallis or Kolmogorov-Smirnov non-parametric test, *p<0.05, **p<0.01. Results are shown as means±SEM. n=3 or 4/group for FIGS. 10A-10C and FIG. 10E and, n=2/group for.

FIGS. 11A-11C. EMAP II mediated SPHK2 signaling promotes local hyperacetylation of histone H3K9 of KLF4 enhancers and alters the local transcription machinery of KLF4 in vascular SMCs. FIG. 11A: Representative immunoblot probed for KLF4, SPHK2 and tubulin in whole cell lysates of hPASMCs following siRNA mediated SPHK2 silencing and post-transfection EMAP II treatment for 6-8 hours, FIG. 11B provides quantitation of KLF4/tubulin and FIG. 11C provides KLF4 expression levels normalized against 18S rRNA in hPASMC cells following siRNA mediated SPHK2 silencing and EMAP II treatment for 6 hours, n=4. Results are shown as means±SEM. Following unpaired t-test or Kolmogorov-Smirnov non-parametric testing, *p<0.05, ****p<0.0001. Results are shown as means±SEM.

FIGS. 12A-12E Vascular ECs can be the endogenous source of EMAP II to initiate SPHK2/Ac-H3K9 mediated KLF4 signaling in vascular ECs. Vascular endothelial cells (ECs) conditioned media (ECM) was collected from ECs grown in 1% O₂ or room air and was used to treat vascular smooth muscle cells (SMCs). FIG. 12A provides representative dot blot probed for secreted EMAP II expression in ECM. FIG. 12B: Representative immunoblot probed for KLF4, SPHK2, tubulin, Ac-H3K9 and total histone H3 in whole cell lysates of normoxia or hypoxia ECM with or without EMAP II neutralizing antibody treated hPASMCs pre-transfected with siRNA mediated SPHK2 or scramble silencing and, FIG. 12C provides quantification of KLF4/Tubulin and FIG. 12D provides quantification of Ac-H3K9/total histone H3. FIG. 12E: KLF4 expression levels normalized against 18S rRNA in normoxia or hypoxia ECM with or without EMAP II neutralizing antibody treated hPASMCs pre-transfected with siRNA mediated SPHK2 or scramble silencing. In summary, EMAP II secreted by vascular ECs promote SPHK2/Ac-H3K9/KLF4 signaling in vascular SMCs that may promote PASMCs proliferation. Following Kruskal-Wallis or Kolmogorov-Smirnov non-parametric test if not mentioned otherwise, *p<0.05. Results are shown as means±SEM and n=3 or 4/group.

FIGS. 13A-13F: EMAP II/SPHK2/Ac-H3K9 mediated KLF4 signaling is a novel pathway that exists in PH disease pathogenesis. FIG. 13A: RNA-seq data of SPHK2, KLF4 and AIMP1 in iPAH: PASMCs and non-iPAH: PASMCs in log 2-fold of count per million (cpm). Following two-way ANOVA, Sidak's multiple comparisons test for logarithmic values. FIG. 13B: Representative immunoblot probed for KLF4, SPHK2, tubulin, Ac-H3K9 and total histone H3 in whole cell lysates of non: iPAH or iPAH PASMCs with scramble or SPHK2 siRNA transfection and, quantification of FIG. 13C Ac-H3K9/total histone H3 and, FIG. 13D KLF4/Tubulin FIG. 13E: KLF4 expression levels normalized against 18S rRNA in non: iPAH or iPAH PASMCs with scramble or SPHK2 siRNA transfection. FIG. 13F: Cell proliferation rate of non: iPAH or iPAH PASMC with or without iSPHK2 pretreatment for 24 hours. The proposed model: Endothelial monocyte activating polypeptide II (EMAP II) plays a key role in reawakening pluripotency factor, KLF4 in human pulmonary artery smooth muscle cells (PASMCs) through stimulation of the nuclear SPHK2/S1P epigenetic modulating axis, suggesting that cooperation between SPHK2 and EMAP II could be a major driving force for epigenetic-mediated vascular PASMCs reprogramming and remodeling in PH. Ablation of SPHK2 expression confers protection against PH by rescuing the global and local transcription machinery from histone acetylation and activation of the pluripotency factor, KLF4. Following Kruskal-Wallis or Kolmogorov-Smirnov non-parametric test if not mentioned otherwise, *p<0.05. Results are shown as means±SEM and n=3 or 4/group.

FIGS. 14A-14H: Pulmonary expression of KLF4 is upregulated in PH. FIG. 14A: Representative immunoblots probed for KLF4 and Tubulin in protein lysates from SPHK2 KO or wild type (WT) control mice (C57BL/6NJ) were subjected to 3 wks of hypoxia (10% 02) or normoxia (room air) and, FIG. 14B presents quantification of KLF4/Tubulin. FIG. 14C presents KLF4 expression levels normalized against Hprt1 utilizing cDNA synthesized from RNA extracts of SPHK2 KO or wild type (WT) control mice that were exposed to 3 wks of hypoxia (10% 02) or normoxia (room air). FIG. 14D: KLF4 expression levels normalized against 18S rRNA in human iPAH or FDL, n=20/group. FIG. 14E: Representative immunoblot probed for KLF4 and Tubulin in protein lysates of human iPAH or FDL tissue specimens and FIG. 14F presents quantitation of KLF4/Tubulin in protein lysates of human iPAH or FDL, n=20/group. FIG. 14G: Representative immunoblot probed for OCT4A and tubulin in hPASMCs following EMAP II treatment for 0, 1. 2, 4 and 6 hours and quantitation of OCT4A/Tubulin. FIG. 14H: Representative immunoblot probed for SOX2 and tubulin in hPASMCs following EMAP II treatment for 0, 1. 2, 4 and 6 hours and quantitation of SOX2/Tubulin. Following Kruskal-Wallis or Kolmogorov-Smirnov non-parametric test or one-way ANOVA, Tukey's multiple comparisons test, *p<0.05, ****p<0.0001. Results are shown as means±SEM and n≥3/group.

FIGS. 15A-15C: SPHK2 mediated histone H3K9 acetylation in PH vascular PASMCs. FIG. 15A: SPHK2 expression levels normalized against 18S rRNA in iPAH: PASMCs and non-iPAH: PASMCs. FIG. 15B: Quantification of SPHK2/Tubulin in FIG. 8H, FIG. 15C: SPHK1 expression (log of counts per million) in iPAH: PASMCs compared to non-iPAH: PASMCs by RNA-seq in GSE144274. Following Kruskal-Wallis or Kolmogorov-Smirnov non-parametric test, *p<0.05. Results are shown as means±SEM and n≥3/group.

FIG. 16: Hypoxia-induced experimental PH mouse model. FIG. 16 presents data on cardiac output in SPHK2 KO or wild type (WT) control mice (C57BL/6NJ) subjected to 3 wks of hypoxia (10% 02) or normoxia (room air) Results are shown as means±SEM and n≥8/group.

### DETAILED DESCRIPTION

### Abbreviations

Pulmonary arterial hypertension (PAH).
Pulmonary arterial smooth muscle cell (PASMC).
Idiopathic PAH (IPAH).
Pulmonary vascular resistance (PVR).
Histone H3 lysine nine (H3K9).
Acetylated H3K9 (Ac-H3K9).
Sphingosine kinase (SPHK).
Sphingosine 1-phosphate (S1P).
Histone deacetylases (HDACs).
Kruppel-like factor (KLF4).
Endothelial Monocyte Activating Polypeptide II (EMAPII).

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms, or a reduction in a physical characteristic, associated with a specific disorder or condition and/or preventing or eliminating symptoms associated with a specific disorder or condition.

As used herein an “effective” amount or a “therapeutically effective amount” of a drug/cell therapy refers to a nontoxic but enough of the drug/cell therapy to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein an amino acid “substitution” refers to the replacement of one amino acid residue by a different amino acid residue.

As used herein the term “patient” without further designation is intended to encompass any warm blooded vertebrate domesticated animal (including for example, but not limited to livestock, horses, cats, dogs and other pets) and humans receiving a therapeutic treatment in the presence or absence of a physician's supervision.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein “Interfering RNA” or “interference RNA” is any RNA involved in post-transcriptional gene silencing, which definition includes, but is not limited to, double stranded RNA (dsRNA), small interfering RNA (siRNA), and microRNA (miRNA) that are comprised of sense and antisense strands.

### EMBODIMENTS

Obliterative thickening of distal pulmonary artery wall is one of the hallmarks of Pulmonary arterial hypertension (PAH) with remodeling of smooth muscle (SMC) and endothelial (EC) cells being pivotal to vascular muscularization. Current PAH therapy offers only symptomatic relief, targeting vasoconstriction and heart failure but not pathologic arterial wall thickening and remodeling. The initiation and progression of PAH is multi-factorial, with genetic and environmental factors functioning as major drivers of disease progression. Genetic and stochastic influences and environmental stresses can cause the destruction of the epigenetic equilibrium leading to long-lasting effects on development, metabolism and health. Many studies related to PAH focus predominately on genetic modifications leaving a void in our understanding of the contribution that epigenetic regulation has on the evolution of PAH. In general, particularly in humans, underlying mechanisms governing epigenetic reprogramming in PAH remain largely unknown, with fundamental mechanistic insights emerging from experimental model systems.

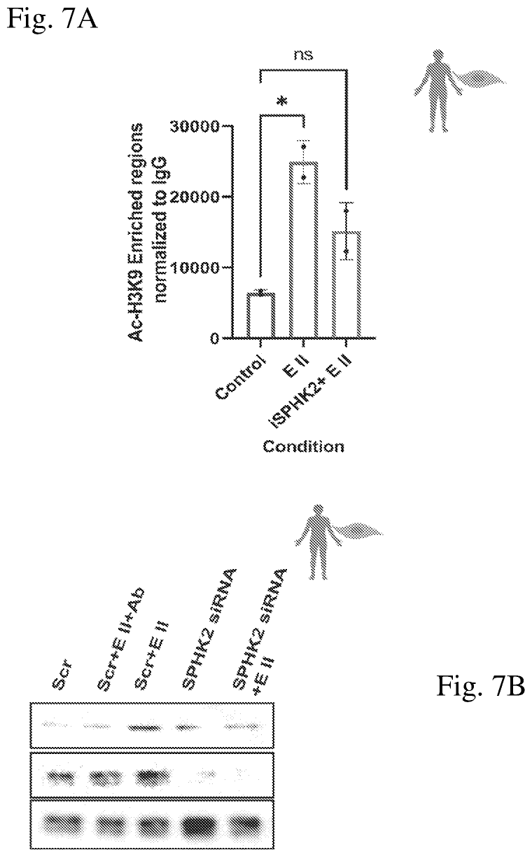

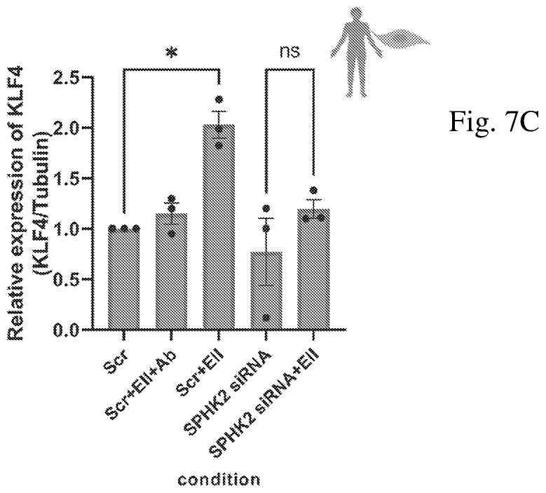

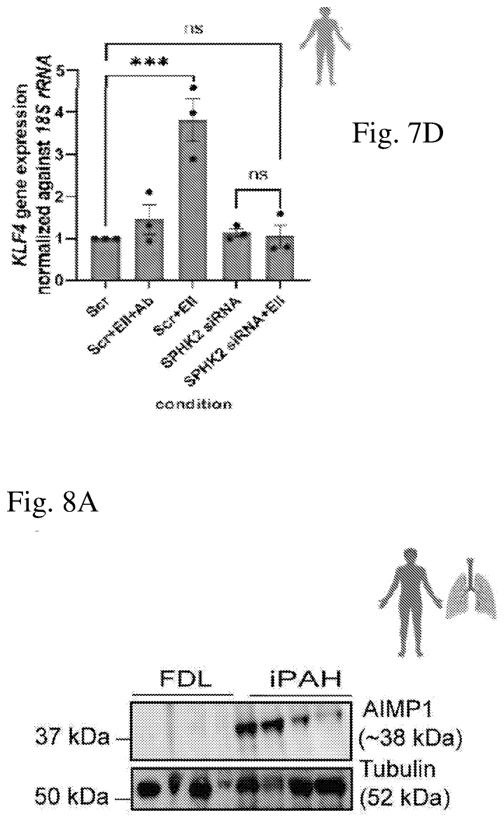

Here, we identify a key role for the SPHK2/S1P axis as a coherent upstream mechanism that cogently explains when, what, and how a disruption of epigenetic equilibrium can occur within diseased IPAH lung. Our studies show that activation of the nuclear SPHK2/S1P axis results in histone H3K9 epigenetic modifications that reawaken PASMC latent gene transcriptions. We demonstrate that the expression of SPHK2, Ac-H3K9 and KLF4 are significantly increased in IPAH patients, identify the mechanistic role that SPHK2/S1P axis has in modulating H3K9 acetylation in PASMC, and determine that KLF4 is a SPHK2/S1P/Ac-H3K9 transcriptional activated target. Moreover, we identify the pro-inflammatory mediator EMAP II as an upstream mediator of the SPHK2/S1P axis that regulates the epigenetic equilibrium via modulating S1P metabolism in PASMC (FIG. 7H). These findings open a new avenue of drug therapy targets for PAH that extend beyond symptomatic PAH treatment.

Covalent chemical changes such as acetylation, methylation and phosphorylation of histone amino-terminal tails have recently identified as key driving forces of vascular biology and disease pathogenesis and are dynamically involved in the regulation of cellular gene expression program. Considering the specific modifications of histone tails or combinations thereof can define the potential transcriptional states of genes. In general, histone acetylation is associated with accessible chromatin structure for transcription whereas methylation can be associated with gene silencing or transcription activation.

Accumulating studies show the importance and mechanism of histone methylation where specifically the hypermethylation of the BMPR2 (bone morphogenetic protein receptor type 2) promoter in the progression of PAH10. However, little is known about the role of chemical modification of histones in PAH pathogenesis. Here, for the first time, we report the significant role of histone acetylation and reactivation of its downstream reprogramming genes in PAH initiation, progression and pathogenesis. Ac-H3K9 is an active chromatin mark of promoter regions and enhancers that has the potential to induce cell proliferation and inflammation.

In breast cancer and lung injury disease processes, previous studies defined the potential of nuclear SPHK2/S1P axis to acetylate H3 and H4 histones. Our studies in human IPAH lungs demonstrate an increase in SPHK2 and Ac-H3K9 levels suggesting a link between SPHK2 and Ac-H3K9 in IPAH. Accumulating studies demonstrated a link between epigenetic modifications such as histone acetylation and the regulation of gene expression patterns that influence the development, metabolism and pathogenesis of disease processes. This reversible modification of acetylation of lysine residues is enzymatically regulated and catalyzed by histone acetyl transferases (HATs) resulting in the addition of acetyl groups to the histone tails to facilitate the opening of chromatin spatial structures and the activation of transcription. In contrast, the histone deacetylases (HDAC) actively deacetylate histone tails initiating a closed chromatin formation that represses gene expression. Acetylated H3K9 activates gene transcription responsible for cell differentiation, proliferation and migration, while class 1 HDACs were recently found to be dysregulated in PAH. However, little is known regarding the regulation of these epigenetic mediators on PAH disease progression.

As described herein a sex-based disruption of the epigenetic equilibrium is in part due to increased H3K9 acetylation in human IPAH lung tissues. Importantly, recent studies indicate that sex is a major influencer in the epigenetic reprogramming process of embryonic neural stem/progenitor cells suggesting that hormone expression impacts transcriptome and epigenome of neural stem/progenitor cells. Moreover, HDAC activity has previously been shown to mount specific roles based on estrogen status as in triple negative breast cancer, there is strong evidence for sex based-differential histone acetylation.

Nuclear SPHK2/S1P signaling has been shown to play a critical role in epigenetic regulation of bacterial-mediated inflammatory lung injury, several cardiovascular disease processes and cancer progression. However, this is the first time its significance as an epigenetic modulator in human PAH has been reported. Chen et al. J Cell Physiol. 2020; 235 (1): 141-150 reported that SPHK1 is associated with PAH, but not SPHK2, using human IPAH tissues, PASMC and murine models. However, Chen utilized an antibody generated to a peptide region from the 1-30aa N-terminus that failed to bind to all SPHK isoforms, resulting in the SPHK2a isoform being overlooked. As reported herein, use of an antibody that detects both SPHK2 a and b isoforms showed a significant increase in SPHK2 expression in human IPAH tissues, consistent with a study that showed increased expression of SPHK2 in lung tissues of PAH rat model and increased pulmonary arterial SPHK2 expression in human IPAH lung tissue sections (Pulm Circ. 2016; 6 (3): 369-380 (doi: 10.1086/687766). Moreover, we found that the ablation of SPHK2 in experimental PAH mouse model confers protection against hypoxia induced PAH while opposing the observations reported in the previously mentioned work utilizing SPHK2 KO (SPHK2−/−) mice. There are several possible explanations for these conflicting findings. Specifically, SPHK2 KO mice were generated in C57BL/6NJ background. However, in prior SPHK2 PAH studies the source of the SPHK2 KO mouse is not well defined as the control mice utilized for this study was described as a C57BL6 mouse consistent with terminology for C57BL/6J mice. Utilizing the correct control group for this SPHK2 KO mice is important as there are marked contrasting differences in inflammatory, genetic, and metabolic responses and signatures between C57BL/6J and C57BL/6NJ mice. In addition, it is important to note that SPHK2a is species-specific and only expressed in humans, not in mice, suggesting that it is possible that there could be differences between human and rodent model findings. Taken together, while epigenetic factors drive aberrant gene transcription in PAH endothelial and smooth muscle cells contributing to vascular remodeling, the epigenetic role of SPHK2/S1P axis in PAH has been marginalized.

A key question is where and how this SPHK2 mediated epigenetic reprograming is initiated and caused. S1P is an eminent mediator released by apoptotic cells to attract phagocytes for clearance upon tissue injury. Moreover, a recent study reported that in airway epithelium, Pseudomonas aeruginosa stimulated SPHK2 phosphorylation and nuclear localization to initiate nuclear SPHK2/S1P signaling where it performed a critical role in epigenetic regulation of bacterial-mediated inflammatory lung injury through histone acetylation. Furthermore, in triple negative breast cancer, SPHK2 is associated with HIF and hypoxia mediated cell proliferation. Since nuclear SPHK2 is seen in both inflammation and cell proliferation, SPHK2 has the potential to acts as an alarming molecule of tissue injury and to be associated with tissue repair processes. Hyper-proliferation of pulmonary artery smooth muscle cells (PASMC) is a vital characteristic in PAH pathogenesis leading to prominent intimal thickening and muscularization of the small arterioles. Therefore, PASMC could prove to be a target candidate for SPHK2 as a regulator mediator of cell proliferation.

Moreover, we found that both transcripts and proteins levels of the pluripotency factors KLF4 and SOX2 show a sex-based expression tendency similar to Ac-H3K9 expression pattern in IPAH as female IPAH cohorts showed an increase trend compared to male cohort in KLF4, SOX2 and Ac-H3K9 levels. These findings uncover the importance of unraveling the sex-dependent PAH pathogenesis and could reveal novel therapeutic strategies for PAH. Clinical studies powerfully provide evidence for sex-related epidemiological and pathophysiological differences in PAH patients. PAH has sexually dimorphic features in disease initiation, presentation, and progression. According to patient registry data, women are more susceptible to develop PAH. However, in contrast to the increased frequency of PAH in females, female PAH patients display increased survival compared to their male counterparts suggesting a phenomenon referred to as the “estrogen paradox”. Therefore, the trends in differential pluripotency factor expression and Ac-H3K9 may be the consequences of this so called “estrogen paradox”.

Mechanistic investigations determined that the ectopic expression of OCT4, SOX2 and KLF4 can initiate the reprogramming of somatic cells to induced pluripotent stem cells that closely resemble embryonic stem cells through epigenetic remodeling mechanisms. Furthermore, there is evidence that KLF4 and SOX2 alone without OCT4 also can induce pluripotency. Numerous studies emphasize the role of KLF4 in vascular SMC proliferation and phenotypic switching. In these studies, hypoxia induced endothelial expression of Hypoxia-inducible factor (HIF)-1a/platelet-derived growth factor (PDGF)-r. to prime vascular smooth muscle progenitor cells to express KLF4, a pathway that is in part mediated through histone acetylation. Although our results did not show a significant expression of OCT4 in IPAH lung tissue (data not shown), both KLF4 and SOX2 protein expression and transcription was elevated in the lung tissue extracts from IPAH patients demonstrating a role for pluripotency factors in PAH pathogenesis. In addition, these findings suggest that there is a subset of cells within the distal pulmonary artery region that undergo increased cell proliferation, phenotypic switching and vascular remodeling.

However, little is known about the upstream initiation and modulation of SPHK2 expression or epigenetic activity. Endothelial factors may play an important role as a mediator of SPHK2 epigenetic activity in PASMC as recent studies found that cell-cell contact of EC to hPASMC has a decisive role in driving epigenetic mediated cellular proliferation. Moreover, hypoxia induced endothelial expression of HIF1 and resulted in priming of the vascular smooth muscle progenitor cells to express KLF468,69. We have previously reported the potential role of EMAP II, a pro-inflammatory mediator of vascular growth and remodeling process, in PASMC proliferation leading to PAH and in S1P signaling. EMAP II is proteolytically cleaved from the Aminoacyl tRNA Synthetase Complex Interacting Multifunctional Protein 1 (AIMP1, also known as SCYE-1, p43, and EMAP II) and it has been shown to be expressed in the sub-endothelium of large vessels in quiescent vessels where it functions as a mediator of endothelial cell growth. Furthermore, rEMAP II has previously shown to promote signs of pulmonary hypertension secondarily in a murine model of bronchopulmonary dysplasia. Notably, we observed increased protein expression and localization of EMAP II in a cell layer surrounded with perivascular cells in the distal micro vessels that expressed activated pSPHK2 human IPAH lung sections, suggesting a potential crosstalk between EMAP II and SPHK2 in tissue repair in PAH setting. In this backdrop, we further investigated the role of EMAP II in SPHK2 modulated epigenetic reprogramming in hPASMC. Our data indicate that EMAPII is a potential regulator of SPHK2 expression, phosphorylation and localization into the nucleus of hPASMC. In addition to hPASMC proliferation, these two pivotal biomolecules, EMAP II and SHPK2 may play a critical role in inflammation through histone deacetylation as both EMAP II and the epigenetic activity of SPHK23 have previously shown to be involved in inflammation.

In alignment with our results demonstrating an SPHK2 inhibitor rescued decreased HDAC activity in EMAP II signaling, previous studies using immune-pulldown assays determined that SPHK2 is an integral part of the multi-protein co-repressor complex comprised of the core catalytic components HDAC1 and HDAC2 and p300 (HAT). Within the nucleus, phosphorylation of SPHK2 increases S1P that subsequently results in enhanced gene transcription by functioning as an endogenous inhibitor of HDAC. Previously, in microglial polarization in neuroinflammation studies and metastatic breast cancer studies showed the potential of intra-nuclear SPHK2/S1P axis to reactivate the previously silenced pluripotency factors like KLF4 and OCT4 through histone acetylation. KLF4 has been previously identified as key molecular factor that contributes to the vascular stem cell population through priming differentiated vascular SMCs that results in muscularization of SMCs. Moreover, we showed that EMAP II is capable of inducing PASMC proliferation in part by, SPHK2 expression and activation while downstream of SPHK2 epigenetic role modulated signaling pathway, KLF4 was identified as a prominent target for hyperacetylation by EMAP II. Therefore, future studies will be directed to the exploration of a broader role for EMAP II and its regulatory impact on PASMC in a hypoxic micro-environment known to be associated with PAH.

As disclosed herein the SPHK2/S1P axis has a role in the progression of PAH that has until now been overlooked due to SPHK2's numerous isoform variants and species specificity of isoforms. Furthermore, EMAP II has a key role in reawakening pluripotency factors in PASMC through stimulation of the nuclear SPHK2/S1P epigenetic modulating axis suggesting that cooperation between SPHK2 and EMAPII could be a major driving force for epigenetic mediated vascular PASMC reprogramming and remodeling in PAH. To date, PAH epigenetic research often disregarded sex differences. Importantly, this present study emphasizes the importance of investigating the molecular mechanisms of diseases in a sex-based manner. As female IPAH patients demonstrated greater histone H3K9 acetylation suggesting that they are more susceptible to histone acetylation mediated genetic modifications including pluripotency factor expression. Thus, supporting the premise that could in part account for PAH's high incidence rate in women. In conclusion, these findings are likely to pave the path towards unraveling novel inhibitors that target PASMC remodeling while overcoming the barrier that has previously prevented others from reaching new therapeutic horizons through targeting PAH pathology rather than only symptomatic relief.

Accordingly, while the importance and mechanism of histone methylation in pulmonary arterial hypertension (PAH) are known, previous to applicants' invention, little was known about histone acetylation in PAH pathogenesis. Histone H3K9 acetylation is an active chromatin mark that has been previously reported to be regulated by a nuclear epigenetic regulator, SPHK2. Previously, SPHK2's role in PAH has been overlooked as a result of neglecting it's isoform variants. However, applicants have found a significant role of histone H3K9 acetylation, SPHK2 expression and reactivation of SPHK2/S1P downstream reprogramming of genes in PAH initiation, progression and pathogenesis in IPAH patients and experimental rodent PAH model. SPHK2/S1P axis promotes KLF4 expression through histone H3K9 acetylation in SMCs and can promote SMCs proliferation. Moreover, SPHK2 can regulate HDAC activity in SMCs. Smooth muscle cells (SMC) muscularization in distal vessels is a hallmark of pathogenesis of (PAH) and hyperproliferation of SMCs is a candidate cell population in PAH. Differentiated SMC population can contribute to vascular stem cell population by expressing KLF4. SPHK2 deficiency confers reduced pulmonary vascular resistance, right ventricle hypertension and distal vessel wall thickness. Therefore in one embodiment SPHK2 inhibitors are used to target PASMC remodeling leading to the reversal of a hallmark of PAH pathogenesis; vascular remodeling that will reach new therapeutic horizons through targeting PAH pathology rather than only symptomatic relief.

Acetylated histone H3K9 (Ac-H3K9) and expression of SPHK2 were elevated in lung samples from IPAH patients compared to non-PAH controls with sex-based differential acetylation patterns observed. Utilizing a hypoxia induced experimental PAH mouse model to investigate the role of SPHK2 in PAH pathogenesis, transgenic SPHK2 deficient (SPHK2 KO) mice in low oxygen demonstrated reduced pulmonary vascular resistance and right vascular resistance assessed by non-invasive echocardiogram while distal vessel wall thickness was abated compared to its background control mice. In vitro, in human pulmonary artery smooth muscle cells (PASMC), pro-inflammatory mediator endothelial monocyte activating polypeptide II (EMAP II) treatment promoted SPHK2 activation, expression, and translocation to the nucleus generating nuclear sphingosine 1 phosphate (S1P) and hyperacetylation of H3K9 through regulating HDAC activity via SPHK2/S1P axis. Moreover, nuclear SPHK2/S1P reactivation of pluripotency factor Kruppel-like factor (KLF4) was inhibited by SPHK2 siRNA pretreatment. According to Cleavage Under Targets and Release Using Nuclease (CUT&RUN), EMAP II has the potential to hyperacetylate candidate Cis Regulator Elements (cCRE) of KLF4.

TABLE 1
Inhibitors of SPHKIn

| Inhibitor for | Structure | Vendor |
|---|---|---|
| SPHK1 (PF643) | 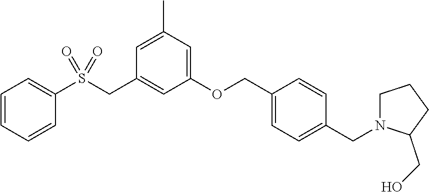 [SMILES: Cc1cc(CS(=O)(=O)c2ccccc2)cc(OCc2ccc(CN3CCCC3CO)cc2)c1] | Cayman |
| SPHK2 (ABC294640) | 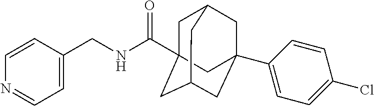 [SMILES: O=C(NCc1ccncc1)C12CC3CC(C1)CC(c1ccc(Cl)cc1)(C3)C2] | Santa Cruz |

In accordance with embodiment 1 a method of decreasing the acetylation of the lysine at position 9 of histone H3 in patients diagnosed with pulmonary arterial hypertension is provided as a method of preventing or treating pulmonary arterial hypertension. The method of decreasing the acetylation of the lysine at position 9 of histone H3 results in reducing the rate of the thickening of distal pulmonary artery wall and/or preventing or reducing the remodeling of smooth muscle (SMC) and endothelial (EC) cells in patients diagnosed with pulmonary arterial hypertension.

In accordance with embodiment 2 the method of embodiment 1 is provided wherein H3K9 acetylation is decreased in a patient diagnosed with pulmonary arterial hypertension by the administration of a pharmaceutical composition comprising an agent that
i) decreases sphingosine kinase 2 activity; or
ii) decreases Kruppel-like factor (KLF4) activity; or
iii) a combination of i) and ii).

In accordance with embodiment 3 a method of embodiment 1 or 2 is provided wherein said method comprises administering an inhibitor of sphingosine kinase 2 to the patient.

In accordance with embodiment 4 the method of any one of embodiments 1-3 is provided wherein the inhibitor is an interfering RNA that targets sphingosine kinase 2.

In accordance with embodiment 5, the method of embodiment 4 is provided wherein the interfering RNA comprises one or more RNAs selected from the group consisting of CAAGGCAGCUCUACACUCA (SEQ ID NO: 1), GAGACGGGCUGCUCCAUGA (SEQ ID NO: 2), GCUCCUCCAUGGCGAGUUU (SEQ ID NO: 3), CCACUGCCCUCACCUGUCU (SEQ ID NO: 4) and complements thereof.

In accordance with embodiment 6 the method of any one of embodiments 1-5 is provided wherein the inhibitor comprises a compound having the general structure:

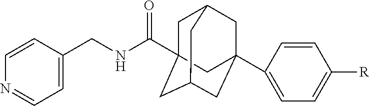

where R is halo.

In accordance with embodiment 7 a method of reducing pulmonary artery vascular remodeling in a patient diagnosed with pulmonary arterial hypertension is provided wherein the method comprises administering an inhibitor of sphingosine kinase 2 to the patient.

In accordance with embodiment 8, a method according to embodiment 7 is provided wherein the inhibitor is
i) an interfering RNA that targets sphingosine kinase 2, optionally wherein the interfering RNA is selected from the group consisting of CAAGGCAGCUCUACACUCA (SEQ ID NO: 1), GAGACGGGCUGCUCCAUGA (SEQ ID NO: 2), GCUCCUCCAUGGCGAGUUU (SEQ ID NO: 3), CCACUGCCCUCACCUGUCU (SEQ ID NO: 4), and complete complements thereof;
ii) a small molecule inhibitor, optionally a compound of the general structure:

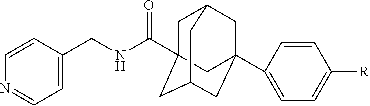

where R is halo, or
iii) a combination of i) and ii).

### Example 1

Role of Acetylated histone H3K9 (Ac-H3K9) and SPHK2 in PAH

### Methods

### Human IPAH and FDL Lung Tissues and EFPE Slides

All studies involving human lung tissues and cells were conducted according to the Institutional review board of Indiana University School of Medicine. Deidentified archived tissue obtained from the Pulmonary Hypertension Breakthrough Initiative (PHBI) tissue biorepository from patients with IPAH at time of lung transplantation and patients without PAH that were failed donor lungs (FDL) included snap-frozen peripheral lung tissues and Ethanol Fixed Paraffin embedded (EFPE) slides.

### Experimental PAH Mouse Model

Animal experimental procedure was performed in accordance with the guidelines issued by the University of Notre Dame/Indiana University Institutional Animal and Use Committee. In the experimental rodent pulmonary arterial hypertension (PAH) model, 12-14 week old mice of SPHK2-deficient [SPHK2 KO] mice in C57BL/6NJ background and C57BL/6NJ (control) mice were exposed to hypoxia (10% 02) in a ventilated chamber or normoxia for 21 days. To avoid possible hormonal issues, only male mice were used.

### Echocardiography of PAH Mice to Non-Invasively Assess Pulmonary Vascular Resistance

The mice were lightly anesthetized using isoflurane anesthesia (with a goal heart rate of 350-450 beats per minute) to perform transthoracic echocardiography. The mouse was placed in the supine position on a temperature-controlled mouse pad. Any hair on the anterior chest was removed. Cardiac function was analyzed via echocardiography using the VisualSonics Vevo 770 ultrasound machine (FUJIFILM VisualSonics, Inc, Toronto, Ontario, Canada). The transducer probe (40 MHZ, VisualSonics Model) was applied to the anterior chest first in a parasternal long axis view, and in B mode setting of the ultrasound machine, a full view of the left ventricle (LV) in the parasternal long axis view can be obtained. Then, the ultrasound was switched to M mode to assess the movement of structures over time. The ultrasound was placed back in B mode and the parasternal short axis view was obtained by placing the transducer probe 90° rotated in clockwise from the parasternal long axis view. The right atrium was focused, and the ultrasound was switched to the color Doppler mode. The pulse wave (PW)-line of the ultrasound was placed over the tricuspid valve to measure flow through the tricuspid valve. Blind-analysis was performed to assess the maximum velocity of tricuspid regurgitation (TRmax velocity) and the velocity time integral of the right ventricular outflow tract (VTIRVOT) using Vevo 770 protocol-based measurements and calculations software. Pulmonary vascular resistance (PVR) was calculated using the following equation.

$$PVR = TR max velocity / VTIRVOT$$

### Assessment of Fulton Index in PAH Mice

Right ventricular hypertrophy was calculated using Fulton index by the weight ratio of the right ventricle (RV) divided by the sum of left ventricle and septum (LV+S). Fulton Index=RV/[LV+S]

### Mouse Lung Microscopy and Morphometry Analysis

Lung tissue sections of 5 microns were prepared. Five-micron sections were subjected to elastin staining and images were taken at 100 micrometer scale. The vascular wall thickness was calculated from elastin-stained sections using the following equation. Wall thickness (%)=(2× medial wall thickness/external diameter)×100 Human Lung Microscopy Analysis Antigens on lung sections of 5 microns were retrieved and stained with pSPHK2 and EMAPII antibodies.

### Cell Culture and Treatments

Primary human pulmonary artery smooth muscle cells (hPASMC) purchased from Lonza (Walkersville, MD) were cultured in complete growth medium containing smooth muscle growth media-2 (SmGM-2) with 10% FBS and growth factors provided as a kit by the supplier (Lonza, (Walkersville, MD). Cells were cultured in a humidified atmosphere with 5% CO₂ at 37° C. For all studies, passages 5-10 were used for hPASMCs. For treatment studies, subconfluent cells plated in multi-well plates were serum starved and if required, pretreated with inhibitors before stimulation with 2 μg/mL recombinant EMAP II. All the controls were handled similar to the test samples. Cells were treated with EMAP II or vehicle, cells were rinsed at the desired time point, and lysis buffer was applied.

### ELISA for S1P Quantification

Equal number of hPASMC cells (15000/cm³) were plated and serum starved for overnight. Cells were pretreated with inhibitors if needed and stimulated for in a series of time points and collected the cells. Cells were pelleted out and washed with PBS. After the extraction of nuclear proteins, protein concentration was measured. S1P levels were measured using S1P ELISA kit (MyBioSource, MBS069092) following manufacturer's instructions. S1P levels were normalized against protein levels.

### Immunoblotting

After appropriate treatments, protein lysates were prepared by using RIPA buffer (for cells) or home-made lysis buffer (tissue) supplemented with phosphatase and protease inhibitors (Thermo Fisher Scientific). Alpha-tubulin or Vinculin was used as a loading control. For nuclear and cytoplasmic fractionation, NE-PER kit from Thermo Fischer Scientific was used. Western blots were performed according to standard methods and quantified using densitometry using Image Studio Lite software from LI-COR Biosciences.

### Immunofluorescence Staining

Primary hPASMCs cells were seeded in Nunc Lab-Tek™ II 4-well imaging plates (Thermo Fisher Scientific) and cultured in growth medium. After the serum starvation cells were stimulated with EMAPII. Treated cells were rinsed with PBS and fixed with 4% paraformaldehyde at room temperature for 15 min and permeabilized with 0.1% Triton X-100 at room temperature for 10 min. After washing with PBS three times, the cells were incubated with pSPHK2 antibody at 4° C. overnight. The cells were then rinsed with PBS three times and subsequently incubated with respective secondary antibody conjugated with Alexa Fluor 647 and Phalloidin 488 at room temperature for 1 h. The cells were rinsed with PBS three times and coverslips were mounted with SlowFade Gold Antifade Mountant with DAPI (Thermo Fischer Scientific) and the cells were examined under Olympus microscope with 40× water objective lens for pSPHK2.

### RNA Extraction and Quantification

Total RNA was extracted with TriZol using Direct-zol™ RNA MiniPrep kit (Zymo Research and reverse transcribed with SuperScript III Reverse Transcriptase (Thermo Fisher Scientific). SPHK2, KLF4, SOX2, 1BS rRNA and Hprt1 transcripts were quantified using PrimePCR SYBR Green assays from BioRad.

### HDAC Assay

Equal number of hPASMC cells (15000/cm3) were plated and serum starved for overnight. Cells were pretreated with inhibitors and stimulated for the desired time points and collected. Cells were pelleted out and washed with PBS. After the extraction of nuclear proteins, protein concentration was measured. HDAC levels were measured using HDAC activity flurometric kit (Abcam, ab156064) following manufacturer's instructions. Free unbound HDAC levels were normalized against protein levels.

### CUTANA CUT&RUN, Illumina Sequencing, and Data Analysis

Cleavage Under Targets and Release Using Nuclease (CUT&RUN) was performed with female hPASMC using CUTANA® protocol v1.6. For each condition, nuclei from two biological replicates were extracted by incubating cells on ice for 10 min in Nuclei Extraction buffer (NE: 20 mM HEPES-KOH, pH 7.9; 10 mM KCl; 0.1% Triton X-100; 20% Glycerol; 0.5 mM spermidine; 1× complete protease inhibitor (GoldBio), collecting by centrifugation (600 g, 3 min, 4° C.), discarding the supernatant, and resuspending at [100 μl/500K nuclei] sample in NE buffer. For each target 500K nuclei were immobilized onto Concanavalin-A beads (EpiCypher #21-1401) and incubated overnight (4° C. with gentle rocking) with 0.5 μg of antibody IgG (negative control), trimethylated H3K4 (positive control), Ac-H3K9 (all validated to SNAP-ChIP nucleosome standards). CUT&RUN enriched DNA was purified and used to prepare sequencing libraries with the Ultra II DNA Library Prep kit (New England Biolabs #E7645S). Libraries were sequenced on the Illumina NextSeq 550, obtaining ˜8 million paired end reads on average. The CUT&RUN paired reads were used for quality control using FASTQ Groomer (Galaxy Version 1.1.5) and paired ends were aligned against the GRCh38/hg38 reference genome assembly for human using bowtie2 (Galaxy Version 2.4.2+galaxy0). After quality-based filtering with SAM or BAM tools (Galaxy Version 1.8+galaxy1), MACS2 (Galaxy Version 2.1.1.20160309.6) was applied for CUT&RUN peak calling of Ac-H3K9 against the IgG control with genome size of 2.7e9 and then, DiffBind (Galaxy Version 2.10.0) was used to analyze the differential binding sites between the control and rEMAP II treated samples.

### Statistical Analysis

The data are presented as means±1 standard error of mean (SEM) from at least three independent experiments if not mentioned. In time-course studies, all the time points were performed on the same day and repeated at least three times over different days. Statistical significance was determined with unpaired Student's t-test or one-way or two-way ANOVA using GraphPad Prism software.

### Results

Pulmonary expression of both an active chromatin mark, acetylated H3K9 and an epigenetic modulator, SPHK2 are elevated in IPAH patients.

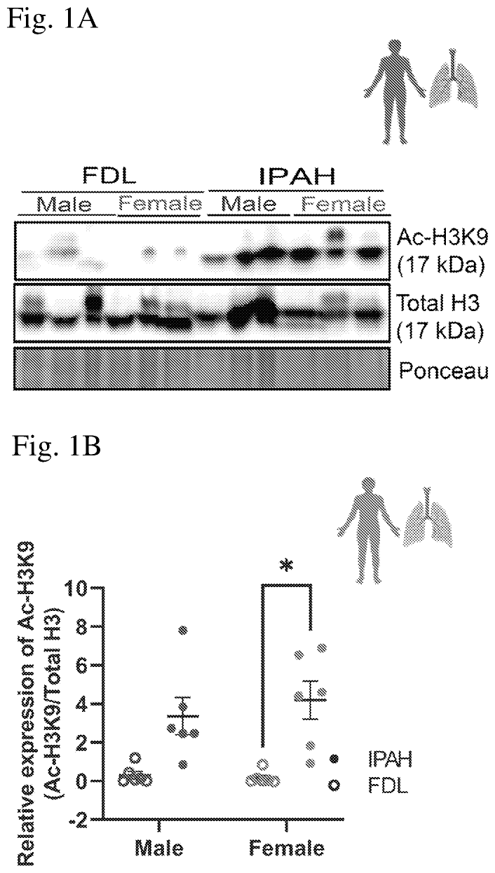

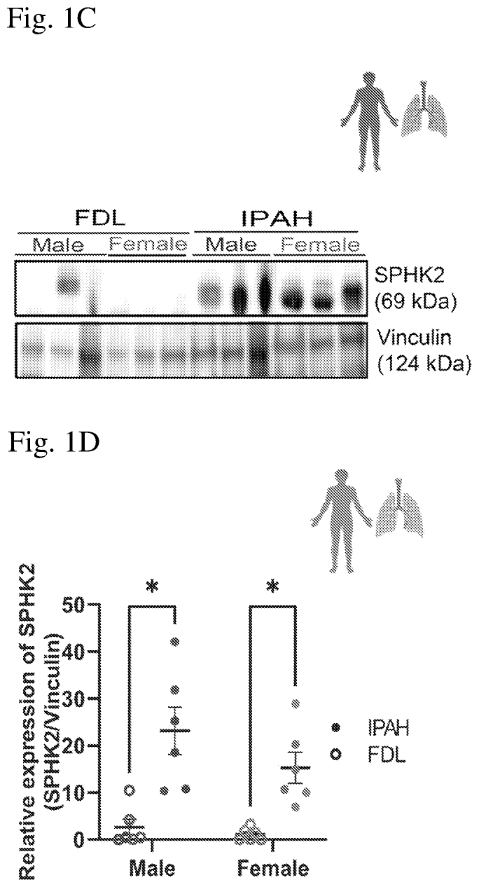

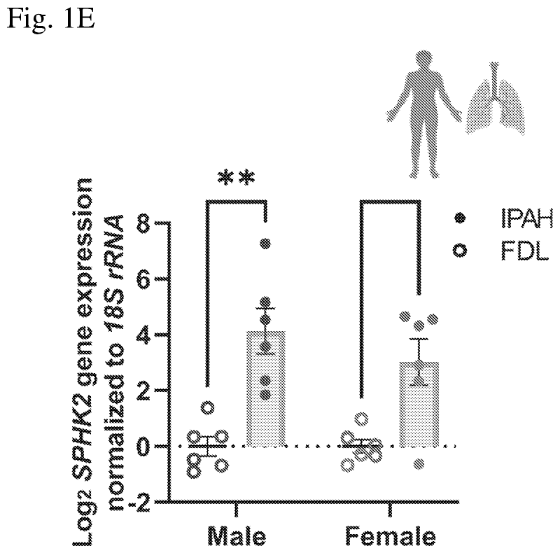

Multifactorial in etiology, the evolution and progression of PAH is mediated by genetic and environmental pressures giving rise to pulmonary vascular muscularization. The few epigenetic studies in PAH have only focused on DNA and histone methylation leaving a gap of knowledge in histone acetylation. Our studies focused on the identification of the role of a key active chromatin mark acetyl-H3K9 (Ac-H3K9) that impacts vascular smooth muscle remodeling. Western blot analysis of Ac-H3K9, determined that H3K9 acetylation levels are elevated in IPAH patient samples with significant increases in Ac-H3K9 levels of female IPAH patient cohort lung tissues as compared to female FDL controls (FIG. 1A). Epigenetic machinery has been implicated as a contributor to the reawakening of latent gene transcription that drives cellular proliferation in PAH. Recent studies identified S1P as a formidable nuclear epigenetic modulator in the other disease processes. However, S1P's epigenetic role and the SPHK2 catalyzing enzyme responsible for nuclear S1P generation in PAH has been overlooked.

Human SPHK2a and b isoforms differ in subcellular location and function. Amino acid (aa) alignment of the isoforms identified that the SPHK2b isoform of 654 aa has a unique N-terminal sequence from aa 1-37, while the SPHK2a isoform is 618 aa and is lacking this N-terminal portion. A previous study that examined SPHK2 in PAH utilized an antibody generated to the N-terminus peptide region of 1-30 aa resulting in other isoforms (e.g., SPHK2a) being overlooked. Analysis of SPHK2 expression using a C-terminal antibody that recognizes all SPHK2 isoforms and qPCR determined that both protein and transcript levels of SPHK2 are significantly elevated in human IPAH lung tissue specimens (immunoblotting: FIG. 1B and qPCR analysis, FIG. 1C) compared to failed donor lung tissues.

SPHK2 deficiency confers protection against hypoxia induced-PAH pulmonary vascular resistance, right ventricle hypertrophy and vascular remodeling in experimental PAH mouse model.

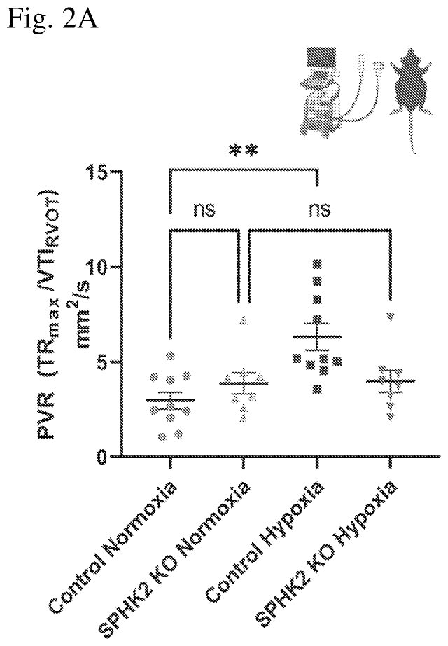

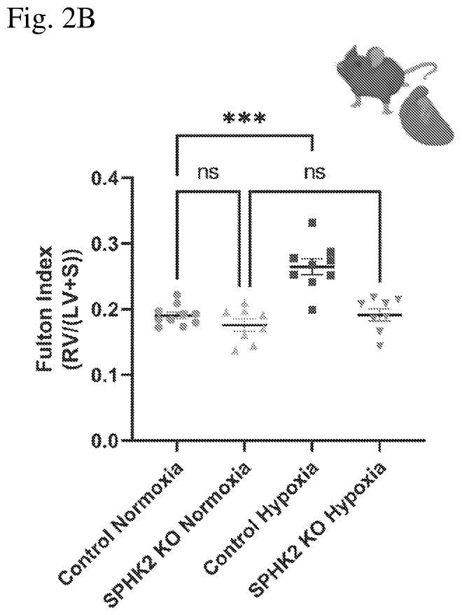

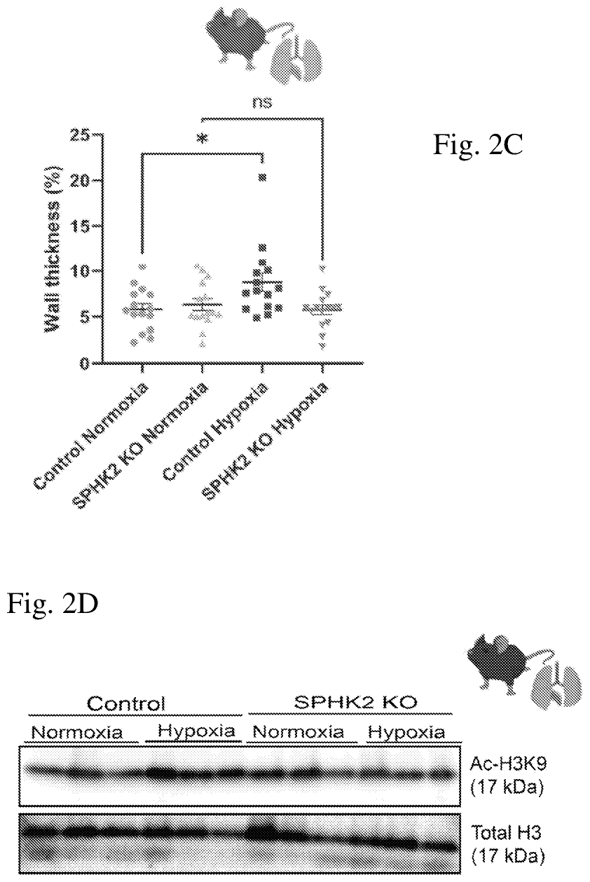

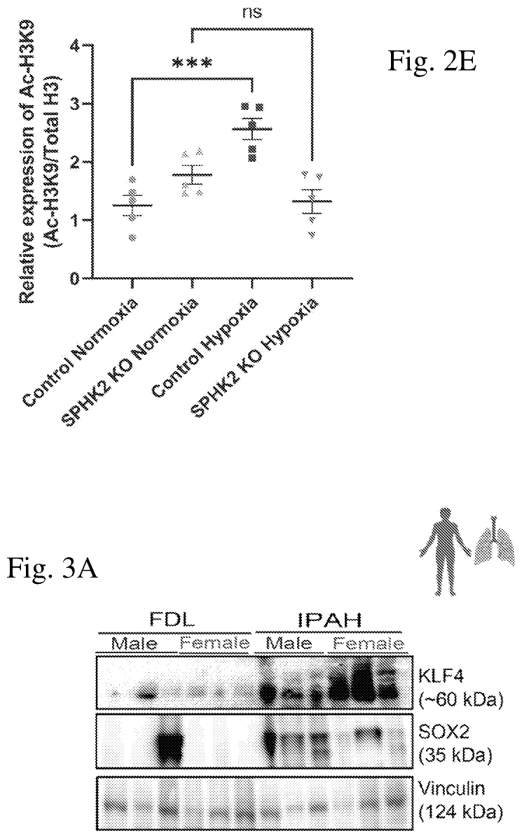

We utilized a hypoxia induced-PAH mouse model by providing a minimal stress of 10% oxygen for 21 days and characterized the model by evaluating three parameters, pulmonary vascular resistance, right ventricle hypertrophy and distal vascular remodeling. C57BL/6NJ mice exposed to 10% O₂ (control hypoxia) showed elevated pulmonary vascular resistance (PVR), (assessed by non-invasive echocardiography), right ventricle hypertrophy (assessed by fulton's index) and distal vessel wall thickness (assessed by histology studies) compared to the C57BL/6NJ mice exposed to 21% O₂ (control normoxia) (FIGS. 2A, 2B and 2C). SPHK2 KO mouse in hypoxia (SPHK2 KO hypoxia) demonstrated no change with cardio-pulmonary parameters were similar to SPHK2 KO normoxia mice, while also demonstrating a reduced pulmonary vascular resistance, right ventricle hypertrophy and vessel wall thickness compared to its background control hypoxia mice (FIGS. 2A, 2B and 2C). Moreover, hypoxic C57BL/6NJ control mice showed increased expression of Ac-H3K9 while ablation of SPHK2 (SPHK2 KO) in hypoxia lowered the hyperacetylation in mice (FIG. 2D) suggesting that SPHK2 deficiency may offer protection against PAH through rescuing disrupted epigenetic equilibrium of H3K9 acetylation.

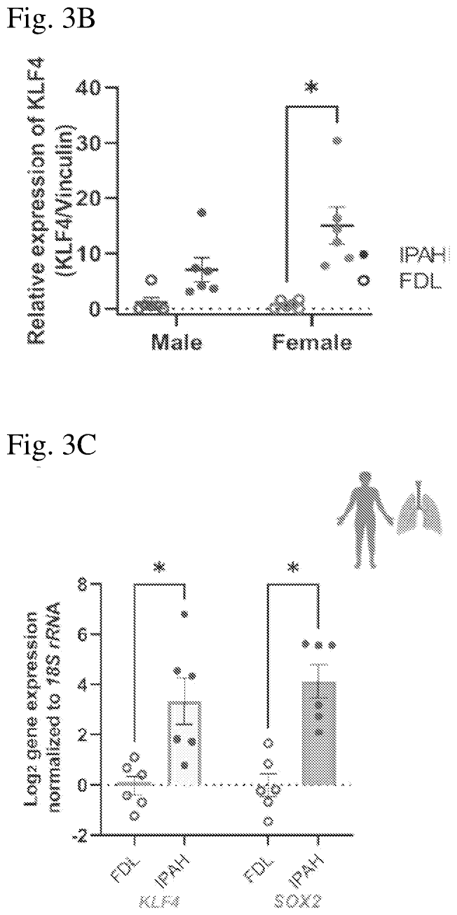

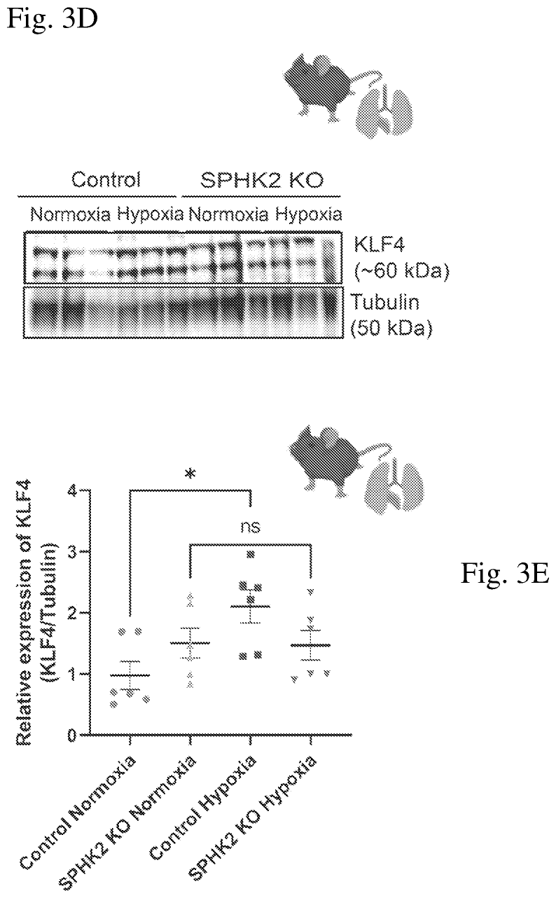

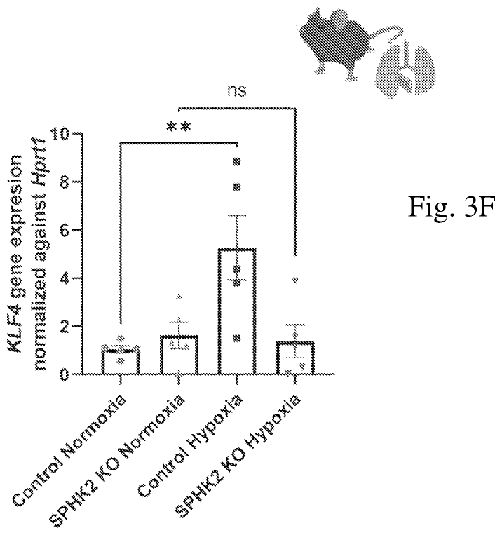

Reawakening of latent pluripotency factor KLF4 is a potential downstream epigenetic target of nuclear SPHK2/histone H3K9 acetylation pathway in lung tissue of IPAH patients and hypoxia-induced experimental PAH mouse model. Nuclear SPHK2 gives rise to nuclear S1P that can bind to the histone deacetylases to release the HDAC co-repressor complex resulting in chromatin relaxation and gene transcription. Therefore, we assessed the transcription levels and protein levels of pluripotency factors KLF4 and SOX2 in human IPAH tissue and experimental PAH mouse model. Reawakening of KLF4 and SOX2 can reprogram differentiated somatic cells into induced pluripotent stem cells (iPSCs). An increasing trend of protein levels (FIG. 3A) and transcript (FIG. 3B) of hyperacetylation sensitive downstream targets: KLF445 and SOX246 were observed in human IPAH lung tissue extracts compared to FDL. Consistent with sex-based differential acetylation of H3K9 in IPAH lung tissues, elevation of protein expression was only significant in female IPAH patient cohort suggesting a sex-based differential sensitivity to acetylation mediation and differential pluripotency factor response in IPAH. Similar to human data, experimental mouse model of hypoxia induced-PAH mouse lung tissue showed increased transcripts and protein levels of KLF4 compared to normoxia mouse lung and SPHK2 deficiency was able to diminish the hypoxia induced KLF4 expression in protein (FIG. 3C) and transcript (FIG. 3D) levels compared to control hypoxia lung tissue.

Pro-inflammatory mediator EMAP II is upregulated in human IPAH lung tissue and induces nuclear phosphorylation of SPHK2 in human pulmonary artery smooth muscle cells.

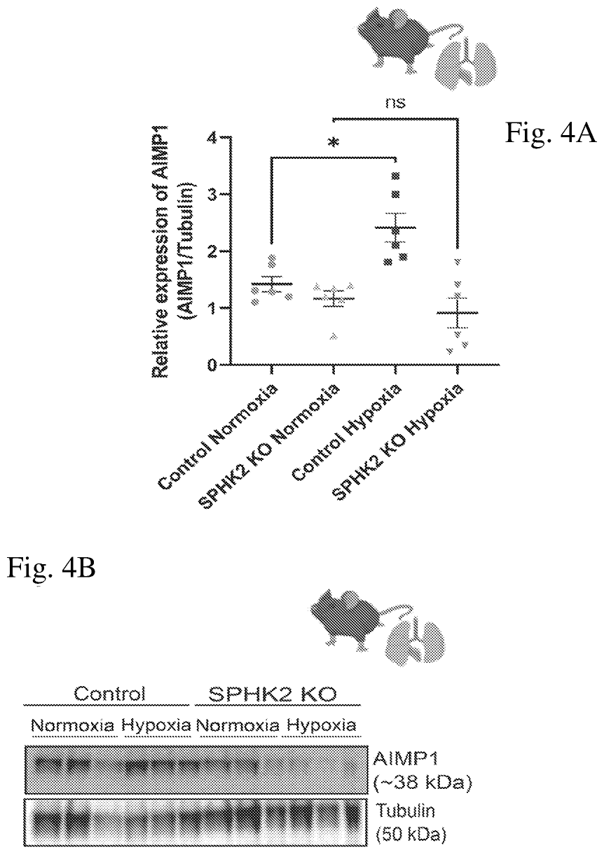

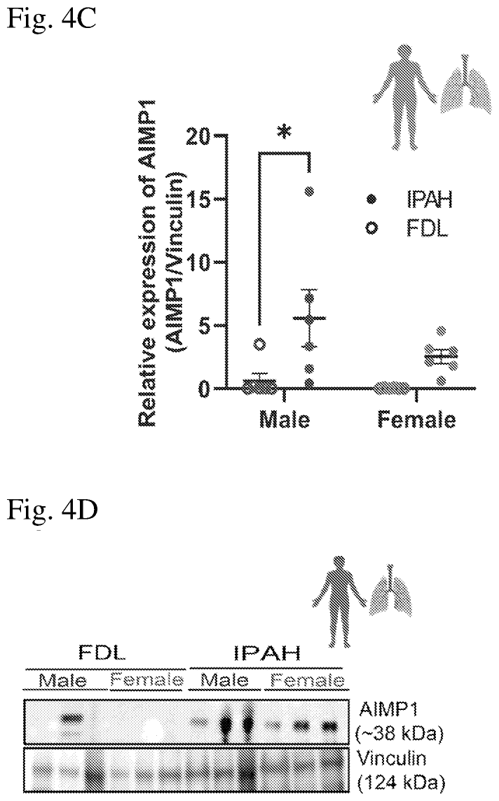

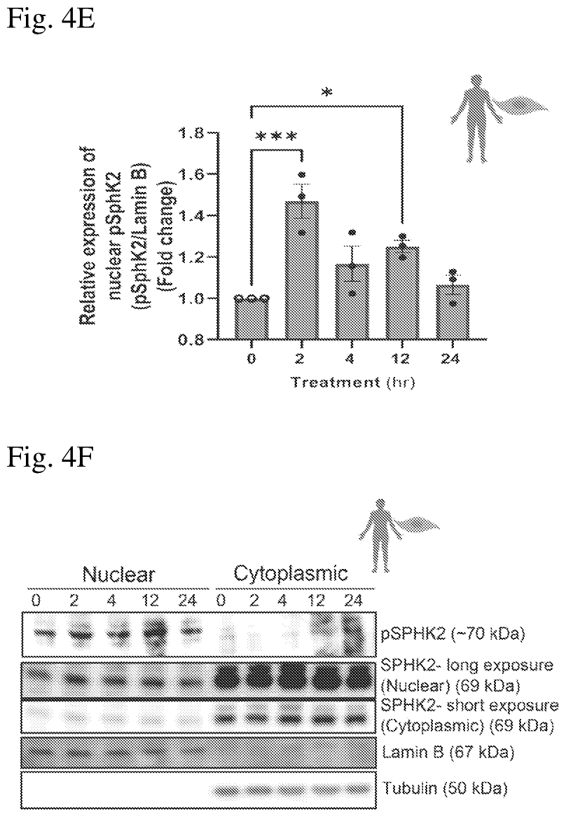

Identification of factors regulating the epigenetic priming of SPHK2 activity in hPASMC cells is important. EMAP II, a known pro-inflammatory mediator of vascular growth and remodeling processes, promotes signs of pulmonary hypertension in a murine model of Bronchopulmonary dysplasia (BPD) while recent in vitro studies determined that EMAP II activation of the SPHK1/S1P axis regulates PASMC proliferation. EMAP II is proteolytically cleaved from the Aminoacyl tRNA Synthetase Complex Interacting Multifunctional Protein 1 (AIMP1, also known as SCYE-1, p43, and EMAP II) and it has shown to be expressed in the sub-endothelium of large vessels in quiescent vessels where it functions as a mediator of endothelial cell growth. Therefore, we reasoned that EMAPII can initiate SPHK2/S1P mediated histone H3K9 acetylation signaling pathway. In experimental PAH rodent models, the precursor form, AIMP1 is overexpressed in hypoxic C57BL/6NJ control mice but suppressed in SPHK2 KO hypoxic mice (FIG. 4A) suggesting a feedback communication between SPHK2 and AIMP1. AIMP1 protein expression in IPAH lung tissue was increased as compared to FDL (FIG. 4B). Immunohistochemical analysis determined that EMAP II expression was elevated in microvascular regions within the thickened remodeled vessels of IPAH lungs. Adjacent to the EMAP II expressing cells in IPAH tissue, cellular expression of nuclear pSPHK2 was prominent suggesting a potential interaction between EMAP II and phosphorylation of SPHK2. Historically, hyper-proliferation of pulmonary arterial smooth muscle cells (PASMC) is a vital characteristic in PAH pathogenesis leading to prominent intimal thickening and muscularization of the small arterioles. As EMAP II expression was peri-adjacent to pSPHK2, the impact of EMAPII in SPHK2's activity in human (h) PASMC was explored. Immunoblotting of subcellular fractions determined that recombinant (r) EMAP II treated hPASMC induced a significant increase in nuclear localized activated SPHK2/pSPHK2 after 2 hours and 12 hours (FIG. 4C).

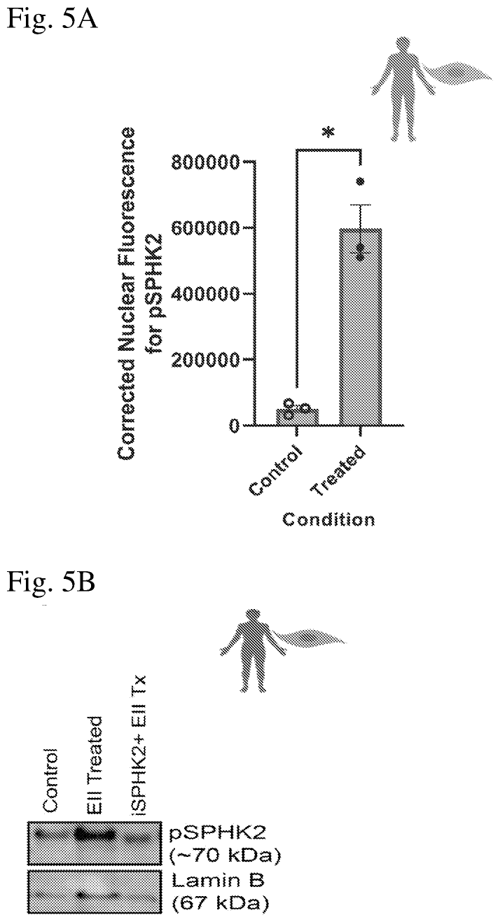

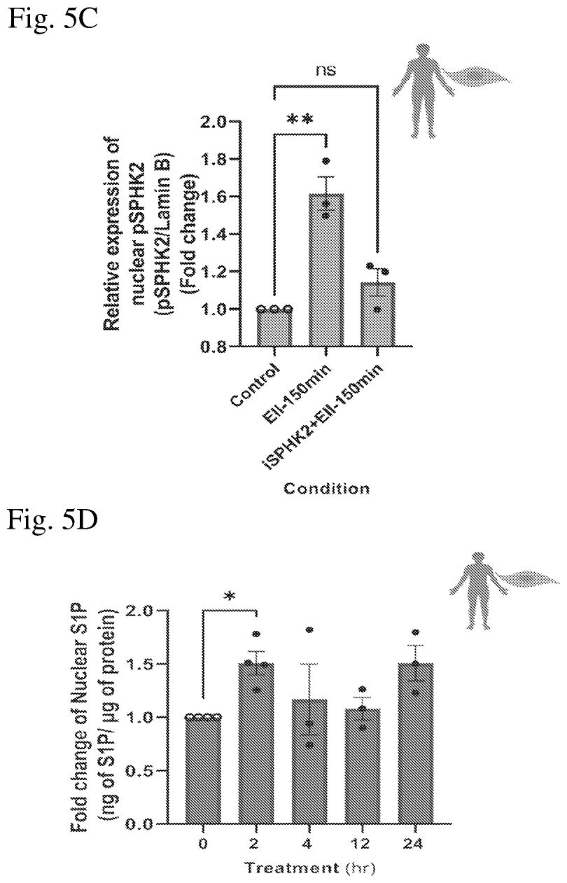

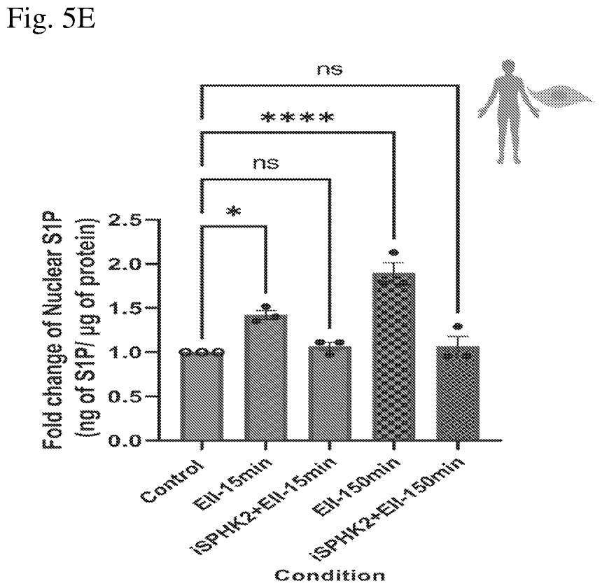

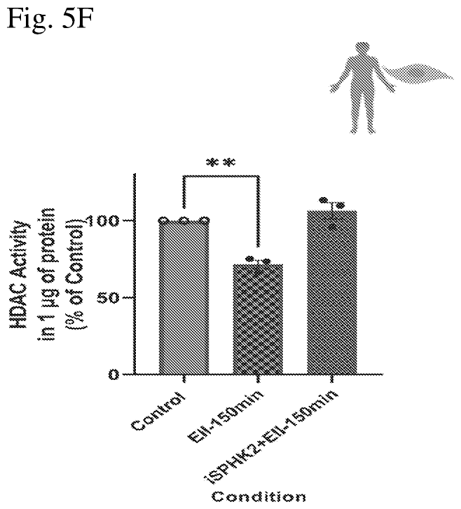

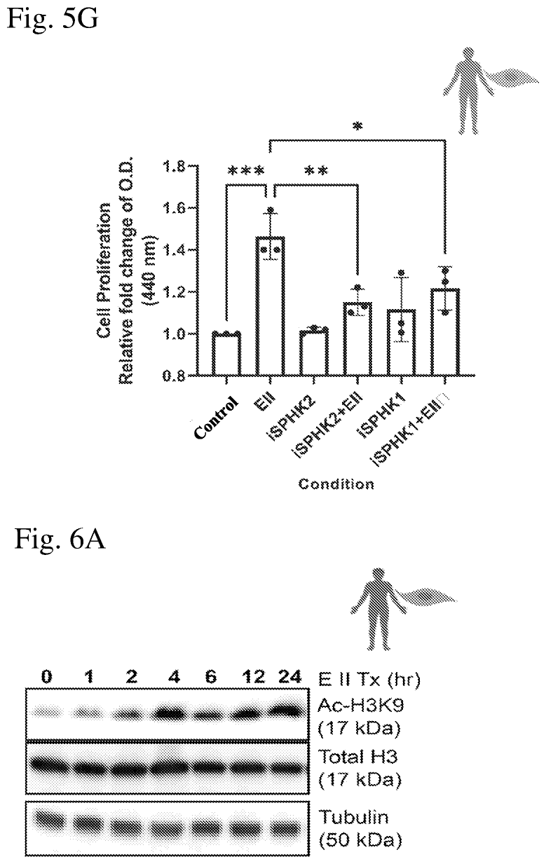

EMAP II mediated nuclear SPHK2 increased nuclear S1P, decreased HDAC activity and induced proliferative SMC phenotype. Immunofluorescence confirmed EMAP II induction of nuclear pSPHK2 in hPASMC with pSPHK2 signal superimposed with the nucleus stained with DAPI, while only a minimal amount was seen in the cytoplasm (stained for F-actin-green; FIG. 5A). Thus, suggesting that EMAP II induced the nuclear compartmentalization of pSPHK2 while pretreatment with SPHK2 inhibitor ablated the EMAP II induced nuclear activation of SPHK2 (FIG. 5B). Nuclear induction of pSPHK2 is associated with phosphorylation of sphingosine to give rise to S1P. Following rEMAP II stimulation, there was a time dependent elevation of nuclear S1P (FIG. 5C) in hPASMC whereas pretreating with SPHK2 inhibitor suppressed EMAP II generated nuclear S1P expression (FIG. 5D). Furthermore, following rEMAP II treatment there was a significant decrease in the HDAC 1 activity as compared to control while iSPHK2 inhibitor rescued the loss of HDAC activity (FIG. 5E). Importantly, pretreatment of hPASMC with isoform specific SPHK2 and SPHK1 inhibitors, blocked EMAPII mediated proliferation with the SPHK2 inhibitor demonstrating a slightly higher effectiveness (FIG. 5F).

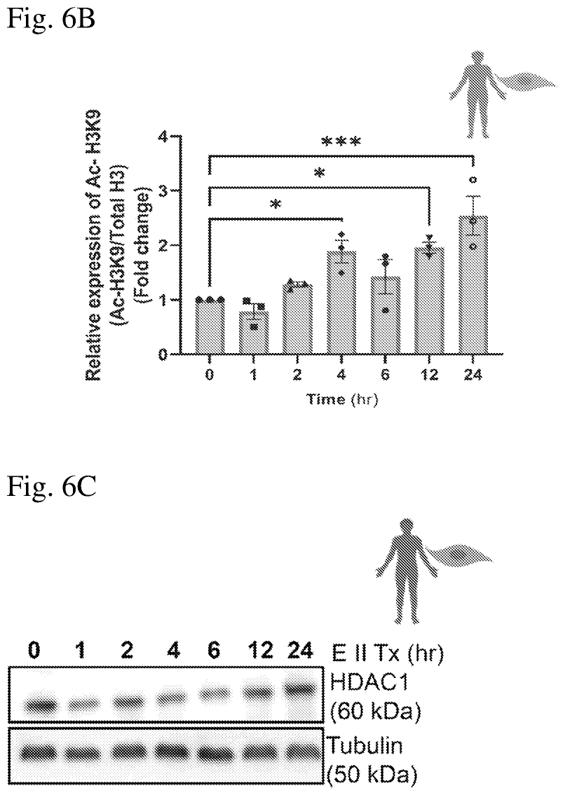

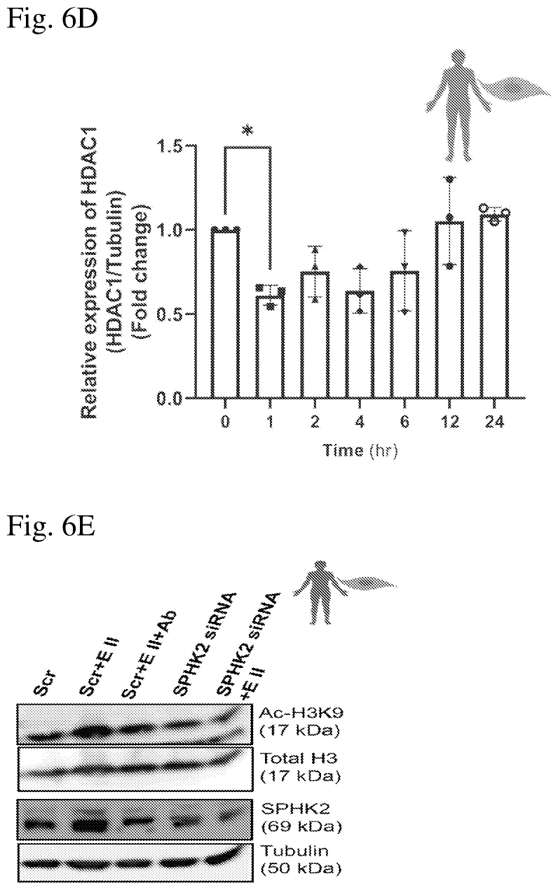

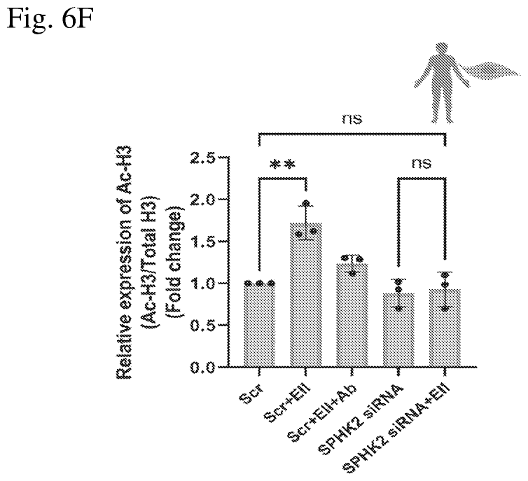

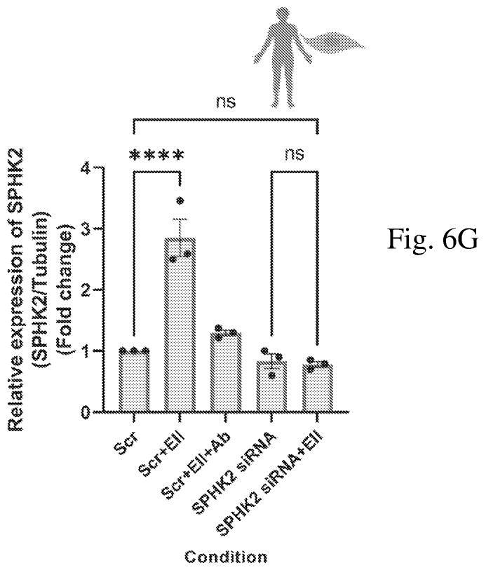

Histone H3K9 acetylation is mediated by EMAP II via SPHK2 in hPASMC. Previous studies link nuclear pSPHK2 and S1P with the acetylation of H3K935. We observed that rEMAP II treatment induced in a dynamic time dependent H3K9 acetylation in hPASMC (FIG. 6A) but not H4K5 acetylation (data not shown). This reversible modification of acetylation of specific lysine residues is enzymatically regulated and catalyzed by histone acetyl transferases (HATs) and histone deacetylases (HDAC). rEMAP II treated hPASMC demonstrated a reduction in HDAC 1 expression (FIG. 6B) consistent with S1P activated histone acetylation and decreased HDAC activity to suppress targeted gene transcription. Thus, supporting a role for EMAP II activation of nuclear S1P to be HDAC mediated Ac-H3K9. Further linking EMAP II induction of nuclear SPHK2 activated S1P acetylation of H3K9, knockdown of SPHK2 using specific siRNA and blocking EMAPII activity by pretreating with an EMAPII neutralizing antibody significantly attenuated rEMAP II induced Ac-H3K9 upregulation at 4 hours (FIG. 6C). Successful knockdown of SPHK2 was confirmed by immunoblotting (FIG. 6D).

EMAP II induction of nuclear SPHK2/S1P axis and acetylation of histone H3K9 modulates hyperacetylation of the KLF4 loci in hPASMC. To determine EMAP II mediated H3K9 hyperacetylation targets in hPASMC, CUT&RUN experiments were performed using an acetylated H3K9 antibody. CUT&RUN analysis of rEMAP II treated hPASMC demonstrated that rEMAP II induced a 2.8 fold hyperacetylated histone H3K9 as compared to the control (FIG. 7A). Importantly, pretreatment with SPHK2 inhibitor partially diminished EMAP II promoted hyperacetylation of H3K9 as 10951 of 11578 (94.58%) of the loci were successfully associated with genes (NIH PAVIS: Peak Annotation and Visualization).

Examination of genes known to modulate vascular remodeling in hPASMC, determined that rEMAP II treatment increased differentially enriched Ac-H3K9 regions in the vascular remodeling gene KLF4 as compared to control. Importantly, pretreatment of hPASMC with a SPHK2 inhibitor rescued the level of Ac-H3K9 regions in EMAPII treated cells suggesting that EMAPII activates KLF4 gene transcription machinery through an SPHK2 mediated hyperacetylation mechanism. Gene annotation using NIH PAVIS showed 5′UTR of KLF4 as an Ac-H3K9 enriched region and moreover, ENCODE candidate Cis-Regulatory Elements overlapped with the enriched regions unique to EMAPII treated CUT&RUN peaks. As KLF4 is frequently implicated in enhancer-dependent transcriptional regulation, EMAPII's role in SPHK2's epigenetic modulation of KLF4 proximal enhancer regions and KLF4 transcription were assessed. Knockdown of SPHK2 expression not only reduced H3K9 acetylation, but ablated EMAP II induction of KLF4 transcription and translation (FIGS. 7B and 7C) after 6-8 hours of rEMAP II treatment.

### Example 2

### Smooth Muscle Cell Histone H3K9 Acetylation in PH

The following experiments were conducted to identify upstream mechanisms that disrupt epigenetic equilibrium of an active chromatin mark, histone H3K9 acetylation in PH pathogenesis. Elevated Ac-H3K9, SPHK2, and pro-inflammatory mediator endothelial monocyte activating polypeptide II (EMAPII) levels were found in human PH samples. Deletion of SPHK2 inhibited hypoxic induced PH and Ac-H3K9 in mice. Uniquely in SMCs, EMAPII altered the acetylome through hyperacetylation of histone H3K9 mediated by nuclear activation of SPHK2 and S1P. CUT&RUN determined that EMAP II has the potential to alter the local transcription machinery of pluripotency factor Krüppel-like factor (KLF4) by hyperacetylating Cis-Regulator Elements of KLF4.

### Methods

Human iPAH (Group 1 PH) and FDL lung tissues and iPAH: PASMCs

All studies involving human lung tissues and cells were conducted according to the Institutional review board of Indiana University School of Medicine. Deidentified archived tissues and pulmonary arterial smooth muscle cells (PASMCs) obtained from the Pulmonary Hypertension Breakthrough Initiative (PHBI) biorepository from patients with Idiopathic Pulmonary Arterial Hypertension (iPAH: Group 1 PH) at time of lung transplantation and patients without PH that were failed donor lungs (FDL).

### Experimental PH Mouse Model

Animal experimental procedure was performed in accordance with the guidelines issued by the University of Notre Dame/Indiana University Institutional Animal and Use Committee. In the experimental rodent PH model, 12-14 wk old male mice of SPHK2-deficient [SPHK2 KO] in C57BL/6NJ background and age-matched C57BL/6NJ [Wild type: WT] male mice were exposed to hypoxia (10% O₂) in a ventilated chamber or normoxia for 21 days.

### Cell Culture and Treatments

Primary human PASMCs (hPASMCs), pulmonary microvascular endothelial cells (hPMVECs) purchased from Lonza (Walkersville, MD) and iPAH: PASMCs were cultured in complete growth medium or conditioned media in a humidified atmosphere or 1% O₂ with 5% CO₂ at 37° C. For all studies, passages 5-10 were used for hPASMCs and hPMVECs.

### Statistical Analysis

The data are presented as means±1 standard error of mean (SEM) from at least three independent experiments, if not mentioned otherwise. Statistical significance was determined with unpaired Student's t-test or one-way or two-way ANOVA for experiments with n≥6 and normality confirmed data sets by Shapiro-Wilk test after log transformation or otherwise Kruskal-Wallis or Kolmogorov-Smirnov non-parametric test for experiments with n<6 using GraphPad Prism software.

### Results

### Pulmonary Expression of an Active Chromatin Mark, Acetylated H3K9, and an Epigenetic Modulator SPHK2 are Elevated in PH Patients.

Our studies focused on the identification and role of a key active chromatin mark acetyl-H3K9 (Ac-H3K9) in vascular remodeling. Western blot analysis of Ac-H3K9 determined that H3K9 acetylation levels are significantly elevated in iPAH patient samples compared to FDL controls (FIGS. 1A & 1B). Ac-H3K9 was significantly elevated in both male and female iPAH patient cohort lung tissues (See FIG. 1B). In other disease processes, recent studies identified S1P as an important nuclear epigenetic modulator through SPHK2 activation of Ac-H3K9. Transcript levels of SPHK2 are significantly elevated in human iPAH lung tissue specimens (FIGS. 1D & 1E). SPHK2a and SPHK2b are the major isoforms that differ in subcellular location and function with the SPHK2b isoform of 654aa demonstrating a unique 1-37 aa N-terminal sequence, while the SPHK2a isoform is 618aa and lacks this N-terminal portion. Despite limitations of high protease and phosphatase activities in procured human iPAH lung samples, analysis of SPHK2 expression using a C-terminal antibody that recognizes all SPHK2 isoforms determined that SPHK2 protein was significantly elevated compared to FDL tissues (SPHK2 antibodies 17096-1-AP, Proteintech and 32346, CST yielded similar results).

### SPHK2 Deficiency Confers Protection Against Hypoxia Induced-PH Pulmonary Vascular Resistance, Right Ventricle Hypertrophy and Vascular Remodeling in Experimental PH Mouse Model.

A hypoxia induced-PH mouse model using 10% oxygen for 21 days was characterized by evaluating PH parameters: pulmonary vascular resistance (PVR) and pulmonary acceleration time (PAT: which inversely and linearly correlates with mean pulmonary arterial pressure (MPAP)) (PVR and PAT were assessed by non-invasive echocardiography), right ventricle hypertrophy (RVH) (assessed by Fulton's index), and distal vascular remodeling (assessed by histology studies). In this study male mice were used, as previous studies indicate that female mice have minimal chronic hypoxia-induced pulmonary hypertensive responses and experience possible fluctuation and interference from ovarian hormones. C57BL/6NJ (WT) mice exposed to 10% O₂ (WT hypoxia) showed elevated PVR, RVH and pulmonary artery muscularization and decreased PAT compared to the C57BL/6NJ mice exposed to 21% O₂ (WT normoxia) (FIGS. 2A-2E). In addition, cardiac output (assessed by non-invasive echocardiography) showed no significant difference among the groups. SPHK2 KO mice in hypoxia (SPHK2 KO hypoxia) demonstrated no change in cardio-pulmonary parameters and were similar to SPHK2 KO normoxia mice, while also demonstrating a reduced PVR and RVH, with rescue of pulmonary artery muscularization and PAT compared to its background WT hypoxia mice (FIGS. 2A-2E). Moreover, hypoxic WT mice showed increased expression of Ac-H3K9 while ablation of SPHK2 (SPHK2 KO) rescued the hypoxia induced hyperacetylation in mice suggesting that SPHK2 deficiency may offer protection against PH through rescuing disrupted epigenetic equilibrium of H3K9 acetylation.

### Pro-Inflammatory Mediator EMAPII is Upregulated in Human PH Lung Tissue and Promotes Histone H3K9 Hyperacetylation in Vascular Smooth Muscle Cells but not in Vascular Endothelial Cells.

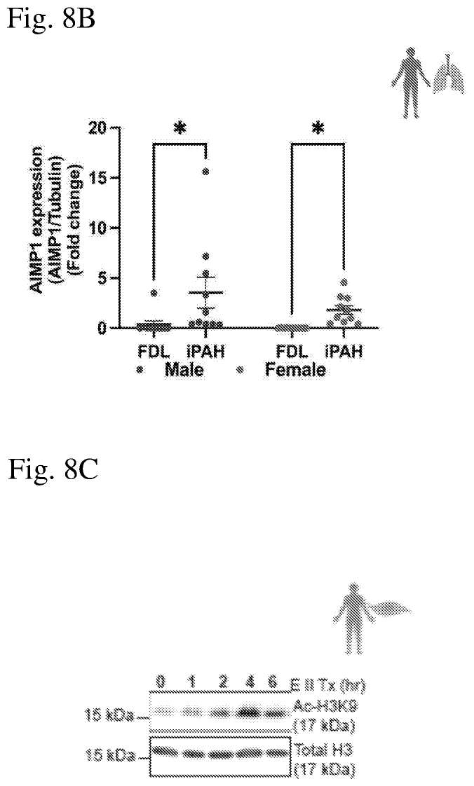

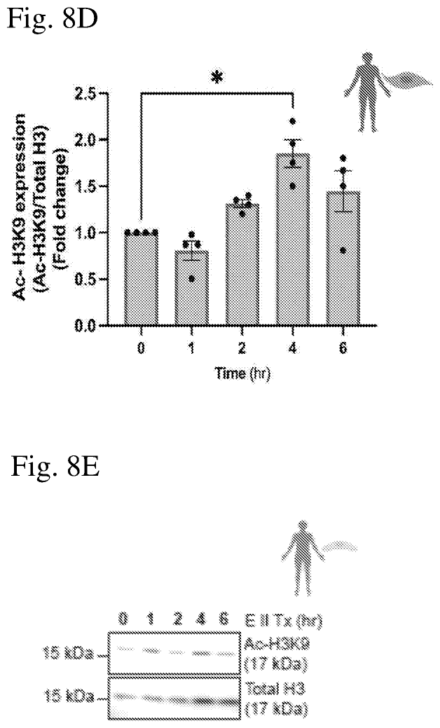

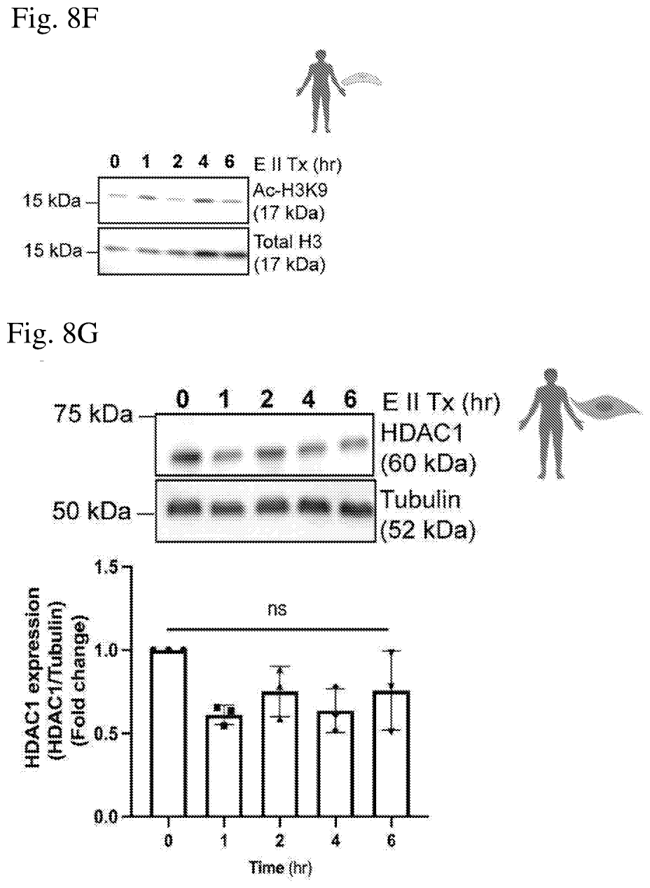

Endothelial monocyte activating polypeptide II (EMAP II), proteolytically cleaved from the Aminoacyl tRNA Synthetase Complex Interacting Multifunctional Protein 1 (AIMP1, also known as SCYE-1 and p43), is a known pro-inflammatory mediator of vascular growth and promotes signs of pulmonary hypertension in a murine model of Bronchopulmonary dysplasia (BPD). In vitro studies determined that EMAPII activation of the SPHK1/S1P axis regulates vascular SMC proliferation. We hypothesized that EMAPII can initiate nuclear activation of the SPHK2/S1P axis. AIMP1 expression was increased in iPAH lung tissue (FIGS. 8A, 8B). Immunohistochemical analysis and previous studies determined that AIMP1 expression was elevated in microvascular regions within the thickened remodeled vessels of iPAH lungs (See FIG. 8G). In mechanistic studies, the potential of EMAPII in histone H3K9 acetylation was examined in two vessel wall cell populations, endothelial cells (human pulmonary microvascular endothelial cells: hPMVECs) and smooth muscle cells (human pulmonary artery smooth muscle cells: hPASMCs). In hPASMCs, recombinant (r) EMAPII treatment induced a dynamic time dependent H3K9 acetylation (FIGS. 8C, 8D) but not H4K5 acetylation (nuclear SPHK2 has previously, reported to increase the acetylation specifically at histone H3K9 and H4K5). Hyperacetylation patterns were not detected in hPMVECs (FIGS. 8E, 8F).

### EMAPII Activates Nuclear SPHK2 and Nuclear Lipid S1P to Promote Histone H3K9 Acetylation and Proliferative SMC Phenotype Via SPHK2.

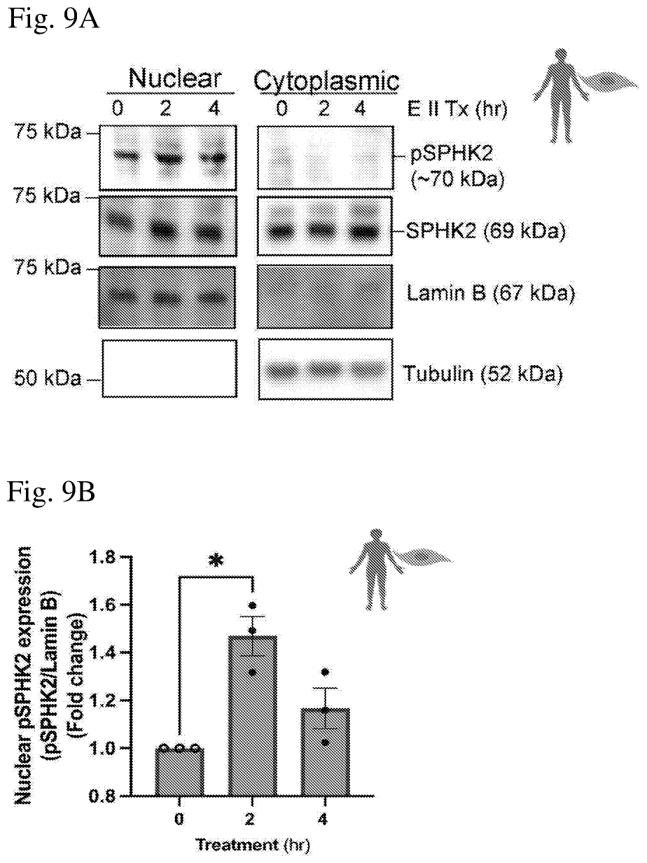

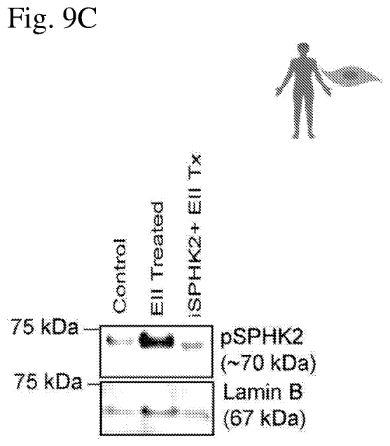

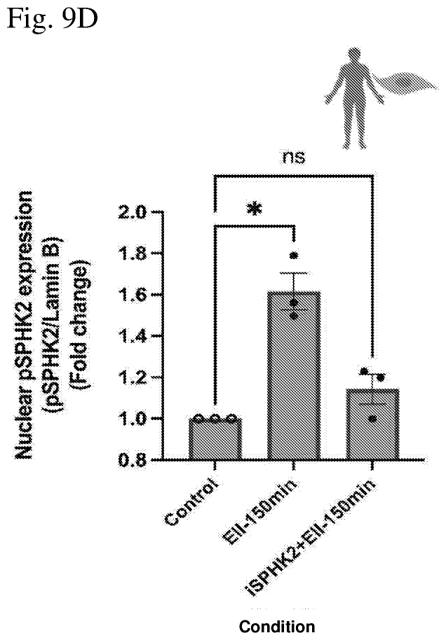

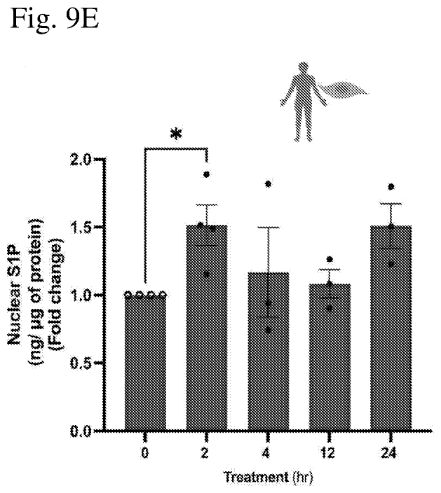

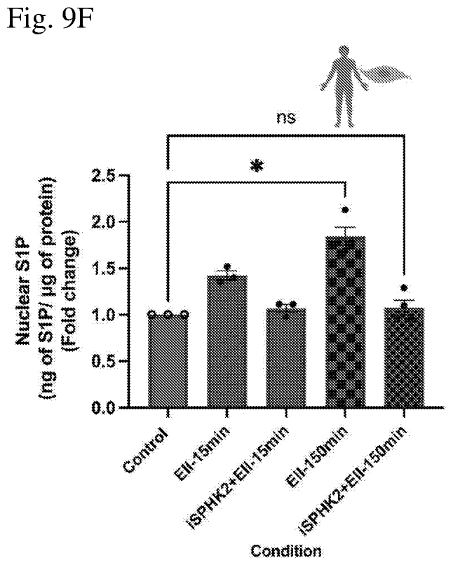

In WT hypoxia mice, we observed increased expression of nuclear pSPHK2 and Ac-H3K9 compared to WT normoxia mice. Furthermore, the prominent co-expression of EMAPII and pSPHK2, suggests a potential interaction between EMAPII and phosphorylation of SPHK2 (See FIG. 8G). rEMAPII treated hPASMCs induced a significant increase in nuclear activated SPHK2/pSPHK2 after 2 hours; but not EMAPII neutralizing antibody pretreated hPASMCs (FIGS. 9A, 9B). Immunofluorescence in hPASMCs confirmed EMAPII induction of nuclear pSPHK2, superimposed with DAPI stained nuclei, with minimal cytoplasmic expression In contrast, in rEMAPII treated hPMVECs, SPHK2 activation was not significant compared to non-treated hPMVECs, where elevated basal expression of pSPHK2 was found in both hPMVEC nuclear and cytoplasmic subcellular fractions of treated and non-treated cells (See FIGS. 9E, 9F) suggesting a more austere role for EMAPII activated SPHK2 in hPASMCs and cell type-specific differential response.

Historically, hyper-proliferation of PASMCs is a vital characteristic in PH pathogenesis. With the significant impact that EMAPII had on nuclear predominant activation of SPHK2 and histone H3K9 hyperacetylation in hPASMCs, our studies focused on EMAPII/SPHK2 mediated epigenetic activity.

Immunoblotting confirmed the EMAPII induced nuclear expression of pSPHK2 while pretreatment with SPHK2 inhibitor (iSPHK2) ablated the EMAPII induced nuclear activation of SPHK2 (FIGS. 9C, 9D). Nuclear induction of pSPHK2 is associated with phosphorylation of sphingosine to give rise to nuclear S1P. Following rEMAPII stimulation, there was a time dependent elevation of nuclear S1P (FIG. 9E) in hPASMCs whereas pretreating with iSPHK2 suppressed EMAPII generated nuclear S1P expression (FIG. 9F). Furthermore, following rEMAPII treatment there was a significant decrease in the HDAC1 activity as compared to control while iSPHK2 rescued the loss of HDAC activity. Unchanged expression of HDAC1/2 supports the previously reported function of nuclear S1P generated by nuclear pSPHK2 as an inhibitor of HDAC activity and promoter of H3K9 acetylation.

### EMAPII Induction of the Nuclear SPHK2/S1P Axis Promotes Global Acetylation of Histone H3K9 in Human Vascular SMCs.

TABLE 1
siRNAs for Knockdown of SPHK2

| SPHK2 | |
|---|---|
| siRNA pool | Sequence |
| 1 | CAAGGCAGCUCUACACUCA (SEQ ID NO: 1) |
| 2 | GAGACGGGCUGCUCCAUGA (SEQ ID NO: 2) |
| 3 | GCUCCUCCAUGGCGAGUUU (SEQ ID NO: 3) |
| 4 | CCACUGCCCUCACCUGUCU (SEQ ID NO: 4) |

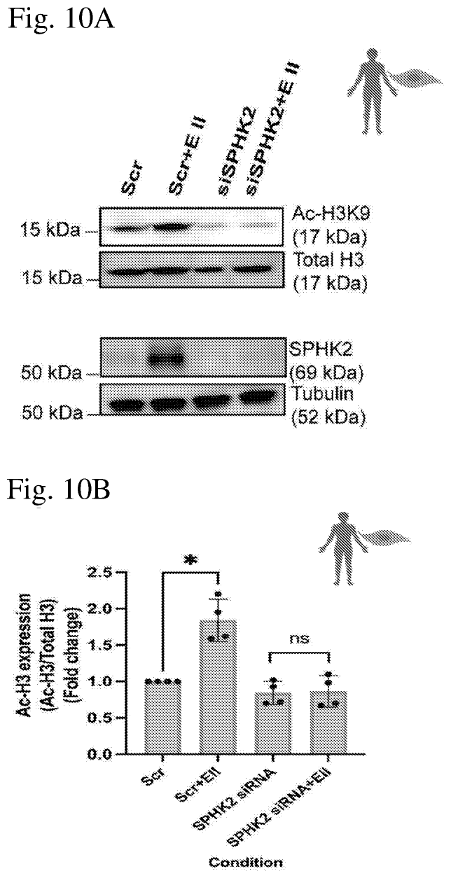

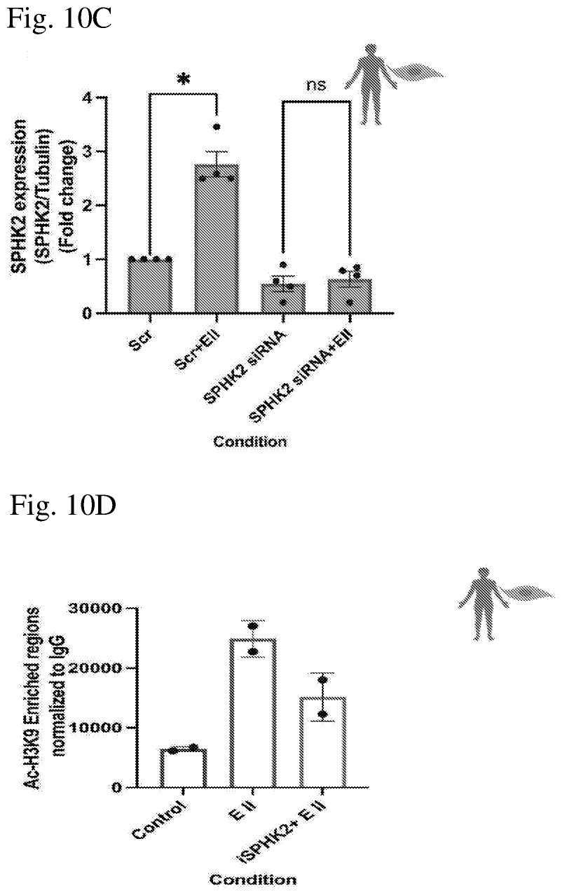

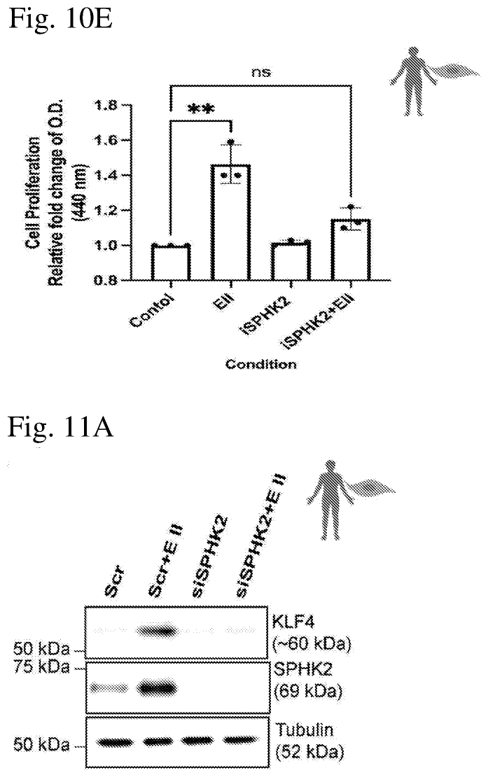

Knockdown of SPHK2 in hPASMCs using specific siRNA significantly attenuated rEMAPII induced Ac-H3K9 upregulation at 4 hours (FIGS. 10A, 10B and successful knockdown of SPHK2 was confirmed by immunoblotting (FIGS. 10A, 10C). This observation further was confirmed and determined EMAPII mediated H3K9 hyperacetylation targets in hPASMCs by CUT&RUN experiments using an acetylated H3K9 antibody. In chromatin mapping by CUT&RUN, two independent biological replicates were used according to the modENCODE repository guidelines. CUT&RUN analysis of rEMAP II treated hPASMCs demonstrated that rEMAPII induced a 2.8-fold hyperacetylated histone H3K9 as compared to the control (FIG. 10C). Pretreatment with iSPHK2 partially diminished EMAPII promoted hyperacetylation of H3K9 (FIG. 10C). Importantly, 10951 of 11578 (94.58%) of the loci were successfully associated with genes (NIH PAVIS), and gene ontology studies identified growth, cellular response to stress and protein modification processes were among the pathways altered by EMAPII mediated hyperacetylation in hPASMCs (ShinyGO v0.75). The role of EMAP II/SPHK2 in cell growth is supported by increased cell proliferation rate in EMAP II treated hPASMCs that is rescued by pretreatment with iSPHK2 (FIG. 10D).

### KLF4 is a Potential Downstream Epigenetic Target of Nuclear SPHK2/Histone H3K9 Acetylation Pathway in PH Patients and Hypoxia-Induced Experimental PH Mouse Model.

Peak annotation and visualization identified 1186 upstream sites out of differentially acetylated sites by EMAPII compared to control that can be responsible for transcription factors binding sites/regulatory elements. Moreover, 5554 differentially acetylated sites by EMAPII compared to control are altered by iSPHK2 pretreatment. The common 755 sites out of differentially acetylated upstream sites by EMAPII compared to control, that is also altered by iSPHK2 pretreatment, suggest the genes that have the highest potential to be transcriptionally regulated by EMAPII/SPHK2 axis through cistrome/epicistrome. The local vascular remodeling gene, KLF4 frequently implicated in enhancer-dependent transcriptional regulation was among these 755 sites

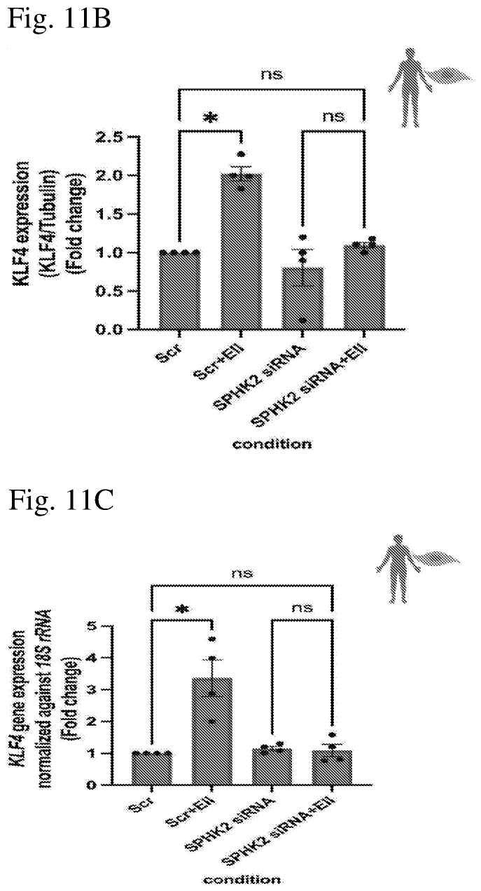

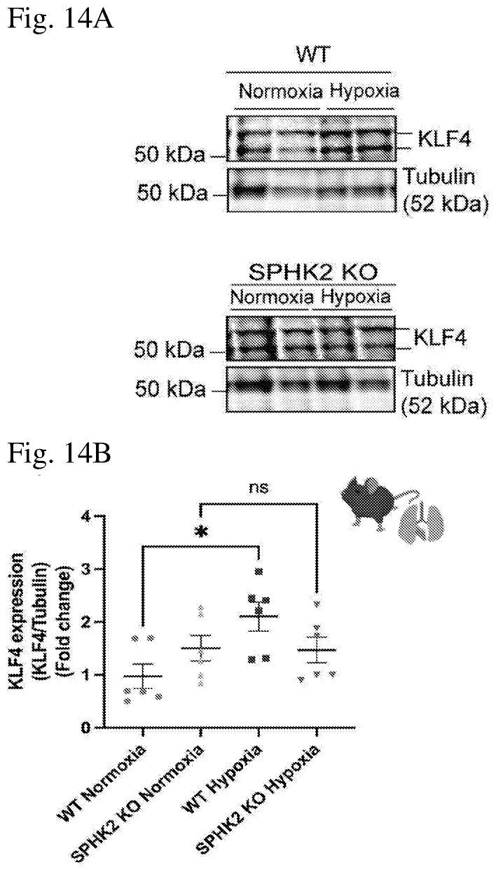

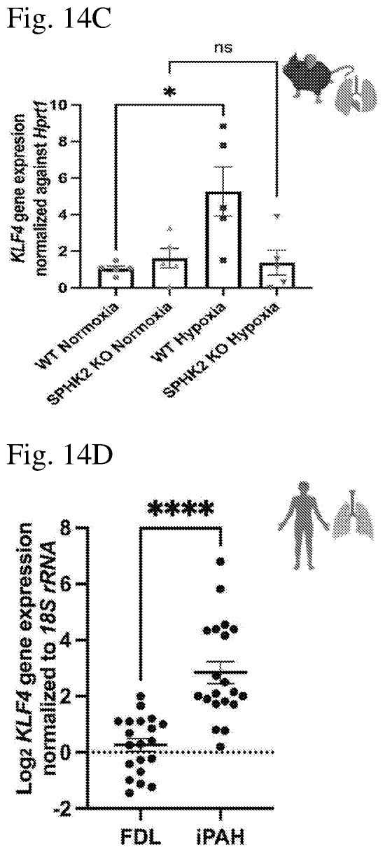

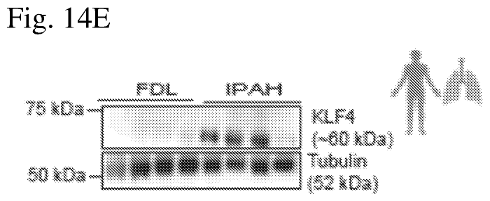

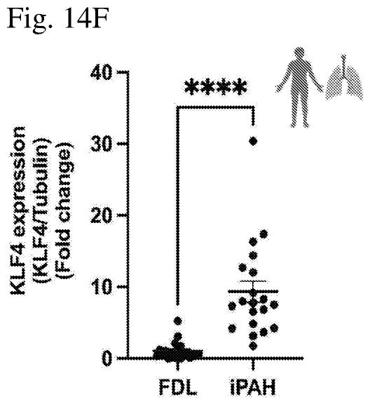

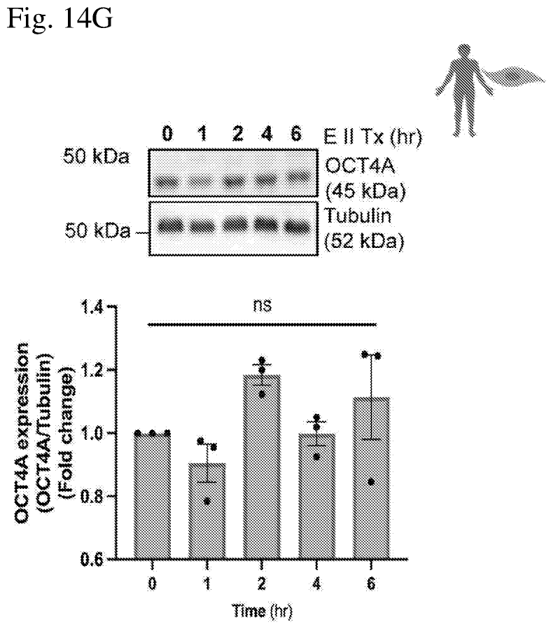

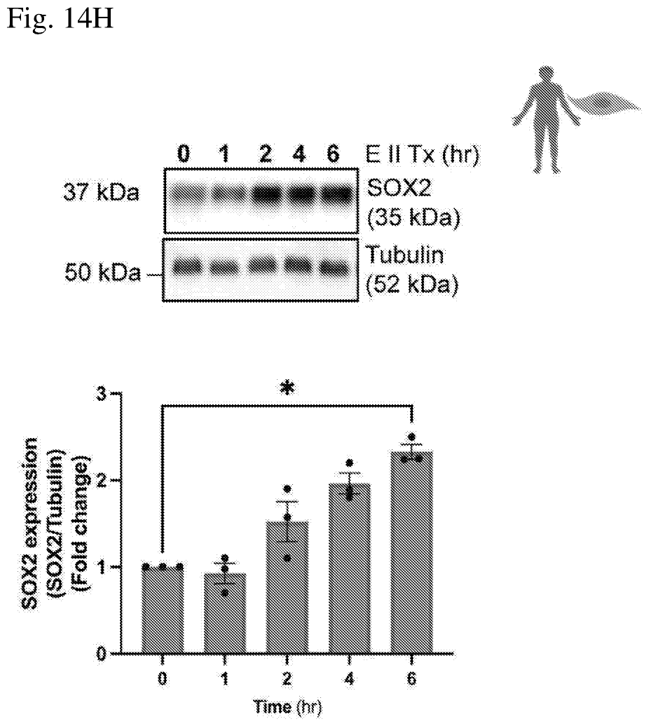

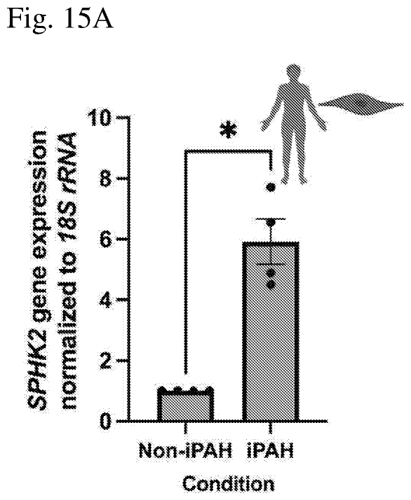

rEMAPII treatment increased differentially enriched Ac-H3K9 regions in KLF4 as compared to control. Notably, pretreatment of hPASMCs with iSPHK2 rescued the level of Ac-H3K9 regions in EMAPII treated cells, suggesting that EMAPII activates local KLF4 gene transcription machinery through an SPHK2 mediated hyperacetylation mechanism. Gene annotation using NIH PAVIS showed 5′UTR of KLF4 as an Ac-H3K9 enriched region and moreover, ENCODE candidate Cis-Regulatory Elements overlapped with the enriched regions unique to EMAPII treated CUT&RUN peaks. Knockdown of SPHK2 ablated EMAPII induced H3K9 acetylation and induction of KLF4 transcription and translation (FIGS. 11A-11C) in PASMCs, suggesting EMAPII/S1P/epigenetics may play a critical role in KLF4 transcription machinery. The transcription and protein levels of KLF4 were increased in human both iPAH lung tissue extracts and experimental hypoxia induced-PH mouse lung tissues compared to their controls (See FIGS. 14A-14F). Moreover, SPHK2 deficiency prevented hypoxia induced KLF4 expression in protein and transcript (See FIGS. 14A-14C) levels compared to WT hypoxia lung tissue.

### Vascular EC Population is a Priming Factor of EMAPII/SPHK2/Ac-H3K9 Mediated KLF4 Pathway in Vascular SMCs.

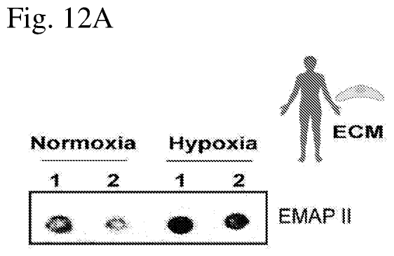

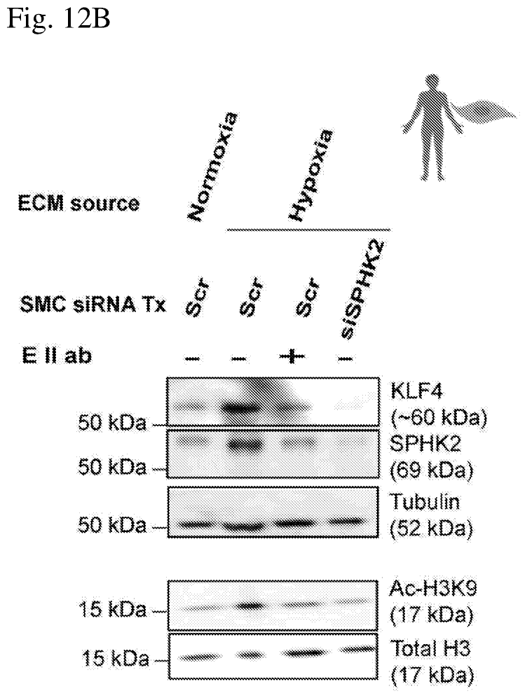

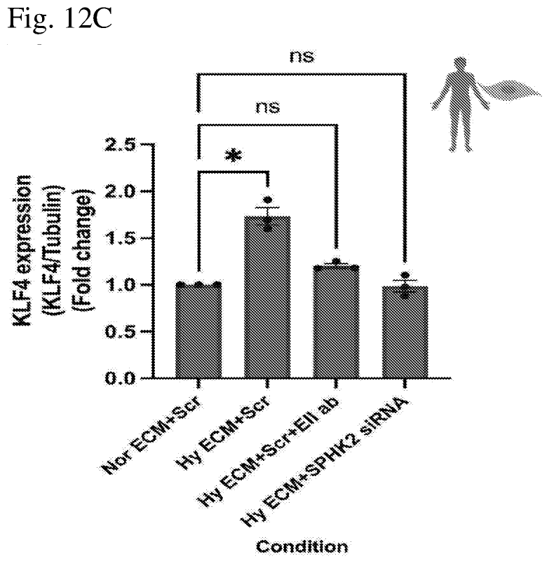

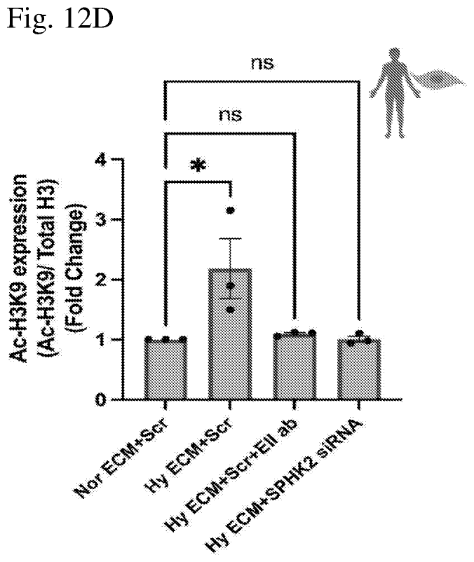

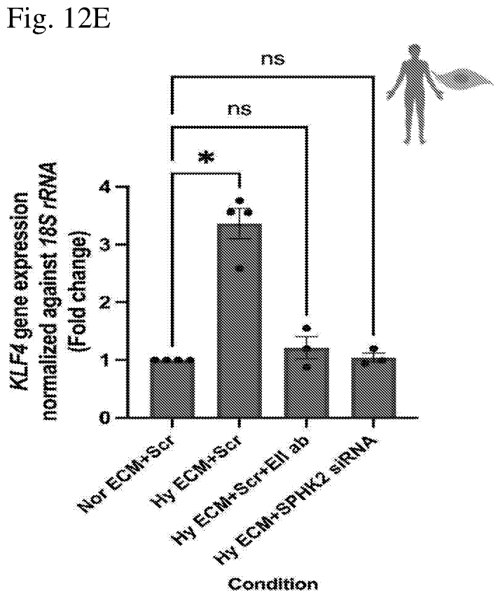

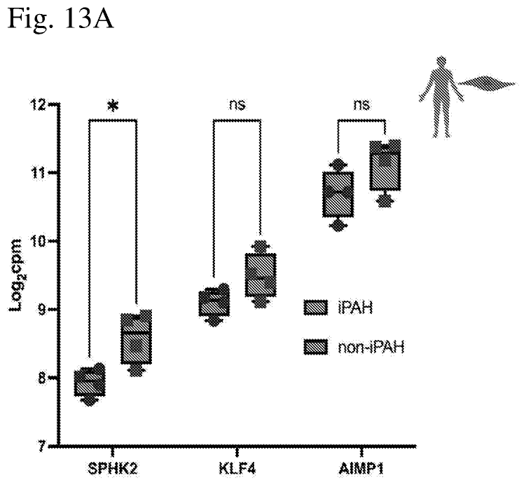

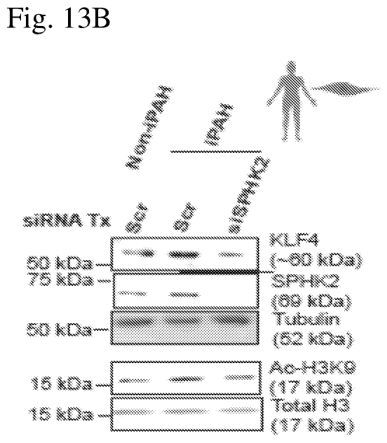

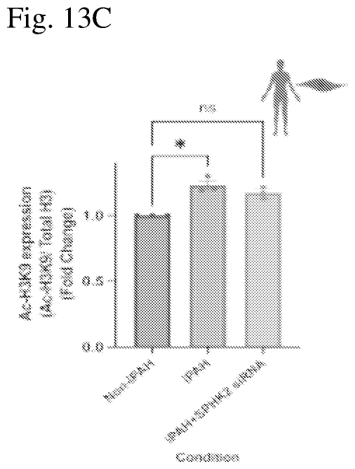

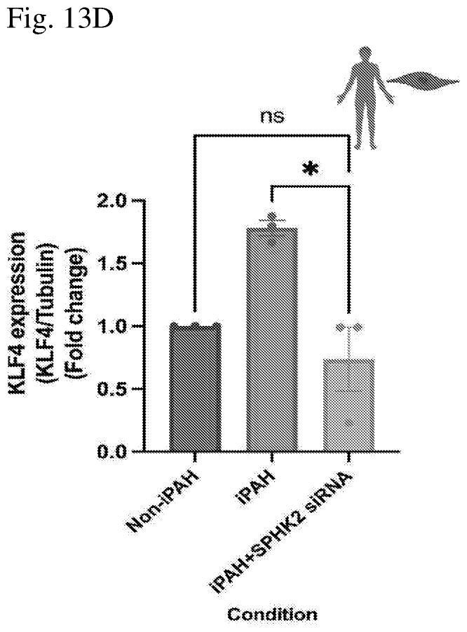

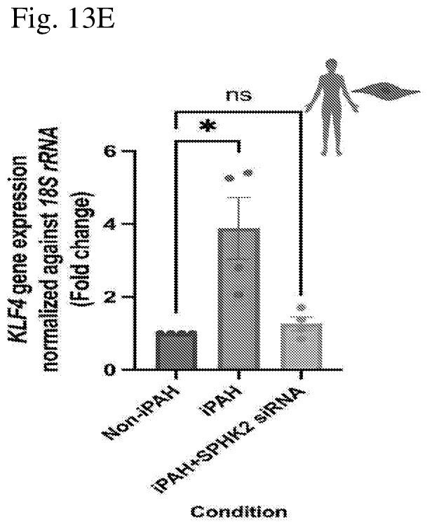

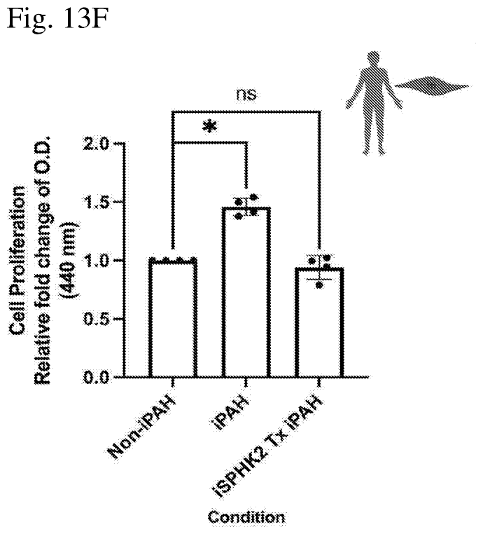

We investigated the potential of ECs to be the endogenous source of EMAPII to initiate SPHK2/Ac-H3K9/KLF4 pathway in SMCs. hPMVEC cultured in normoxia or hypoxia (1% O₂) and cell culture supernatants (endothelial cells conditioned media: ECM) were collected and utilized to treat SMCs. Hypoxia ECM showed an increased expression of EMAPII compared to the normoxia ECM (FIG. 12A). The hPASMCs treated with hypoxia derived ECM demonstrated increased KLF4 expression and Ac-H3K9 levels while preincubation with EMAPII neutralizing antibody restored levels consistent with normoxia ECM treated cells (control) suggesting the vital role of EC secreted EMAPII in SMC's KLF4 expression (FIGS. 12A-12D). Moreover, hPASMCs pretreated with SPHK2 siRNA were resistant to hypoxia derived ECM induction of KLF4 and Ac-H3K9 expression (FIGS. 13C-13G). Thus suggesting a prominent role for endothelial cell secreted factors in activation of the histone acetylome and a contributor to reactivation of the pluripotency factor such as KLF4 through SPHK2.

### EMAP II/SPHK2/Ac-H3K9 Mediated KLF4 Reprogramming in PH: Vascular SMCs.

Cooperatively, the analysis of RNA sequencing of iPAH: PASMC dataset with accession number GSE144274 in NCBI Gene Expression Omnibus (GEO) and qPCR data showed a significant increase in SPHK2 transcripts in iPAH: PASMC emphasizing the important role of SPHK2 in PH that has been previously overlooked (FIG. 13A). Moreover, it showed an increasing trend for both KLF4 and AIMP1 expressions in iPAH: PASMC compared to the non-iPAH: PASMCs (FIG. 8A). The elevated histone H3K9 acetylation (FIGS. 13B, 13C) was partially inhibited by successful knockdown of SPHK2 (FIG. 13B). The increased transcription and expression of KLF4 (FIGS. 13B, 13D and 13E) in iPAH: PASMCs was also inhibited by SPHK2 knockdown. Moreover, the proliferation rate was significantly elevated in iPAH: PASMCs compared to non-iPAH: PASMCs, with iSPHK2 treatment restoring regular proliferation rates in iPAH: PASMCs (FIG. 13F). Altogether, these results validate the potential role of EMAPII/SPHK2/Ac-H3K9/KLF4 signaling in vascular SMCs in PH pathogenesis.

### Discussion

The initiation and progression of PH is multi-factorial, with genetic and environmental factors functioning as major drivers of disease progression. Here, we identify a key role for the SPHK2/S1P axis as a coherent upstream mechanism that cogently explains when, what, and how a disruption of epigenetic equilibrium can occur in PH. Our studies show that activation of the nuclear SPHK2/S1P axis results in histone H3K9 epigenetic modifications that reawaken latent gene transcription of KLF4 in vascular SMCs, but not vascular ECs. Moreover, we identify the pro-inflammatory mediator EMAPII as an upstream mediator of the SPHK2/S1P axis that regulates the epigenetic equilibrium through H3K9 acetylation via modulating S1P homeostasis in vascular SMCs and PH.

Accumulating studies show the importance and mechanistic regulation of global and local histone acetylation code. However, a complete picture of the global histone acetylation code in PH pathogenesis is not available in part because in contrast to stable histone methylation, histone acetylation is very dynamic and transient. Ac-H3K9, an active chromatin mark of promoter regions and enhancers, is an enzymatically regulated reversible modification of acetylation of lysine residues to facilitate the opening of chromatin spatial structures and the activation of transcription. Low-abundant Ac-H3K9 is upregulated upon increased gene transcription, proliferation and inflammation.

Here, we identify how histone acetylation mediated reactivation of its downstream reprogramming gene targets unique to vascular SMCs impacts PH pathogenesis. Upstream of HDACs, Ac-H3K9 has a broad host of downstream targets and could partially account for the conflicting role of targeting HDACs in PH. SPHK2, an integral part of the multi-protein co-repressor complex comprised of the core catalytic components HDAC1/2 within the nucleus, phosphorylation of SPHK2 increases S1P that subsequently enhances gene transcription by functioning as an endogenous inhibitor of HDAC without affecting the activity of histone acetyltransferase.

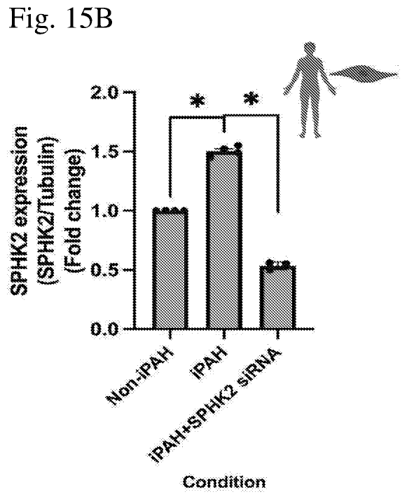

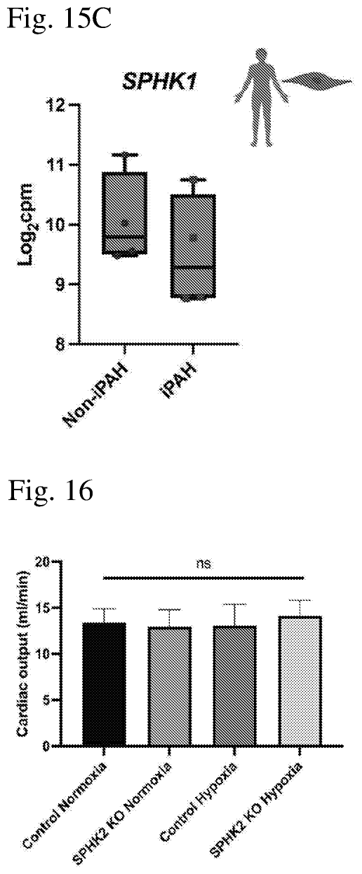

As disclosed herein SPHK2 has functional significance as an epigenetic modulator in human PH collaborating the study that showed increased pulmonary arterial SPHK2 expression in human iPAH lung tissue sections (S G, SR J, MM B, et al. Pulm Circ. 2016; 6 (3): 369-380). Previously, Chen et al (Am J Respir Crit Care Med. 2014; 190 (9): 1032-1043) reported conflicting results as they claimed that only SPHK1 is associated with PH, but not SPHK2. However, this study utilized an antibody generated to a peptide region from the 1-30 aa N-terminus of SPHK2b (long isoform) resulting in the SPHK2a (short isoform) being overlooked. Unbiased RNA sequencing data of iPAH: PASMCs available at the GEO database under GSE144274 showed an increased expression of SPHK2 and no significant change in SPHK1 expression compared to the non-iPAH cells (see FIG. 15C).

An experimental chronic hypoxia-PH mouse model was sufficient for our main investigational focus of mechanisms of pulmonary arteriolar muscularization. The SPHK2 KO mice is generated in C57BL/6NJ background. Chen et al. mentioned control mice in their studies as C57BL6 mice. However, there are marked contrasting differences in inflammatory, genetic, and metabolic responses and signatures between C57BL/6J and C57BL/6NJ mice. In addition, SPHK2a is species-specific, expressed in humans but not in mice, suggesting possible differences between human and rodent model findings. Previously, a protective role by SPHK2 against cardiac fibrosis was reported. However, we did not observe any cardiac function changes in SPHK2 KO mice (See FIG. 16). Defining the epigenetic role of the SPHK2/S1P axis is important in PH.

Little is known about the upstream initiation and modulation of SPHK2 expression and epigenetic activity. Previously we reported that pro-inflammatory mediator EMAPII regulates SPHK1/S1P/S1PR homeostasis through ERK activated SPHK1 to promote hPASMC pro-proliferative factor IL6 but not proinflammatory cytokine TNFα expression. ERK phosphorylates SPHK2 at Ser387 and Thr614. However, Ser387 phosphorylation site sequence is identical to SPHK1. Therefore, our studies focused on the unique phosphorylation of SPHK2 via Thr614. AIMP1/EMAP II expression remains low in postnatal and adult lungs, confined to the sub-endothelium of large vessels in quiescent vessels where it functions as a mediator of endothelial cell growth, while upon vascular insults its expression can be significantly increased. Prior study hints at the possibility that EMAPII promotes signs of PH secondarily in a murine BPD model. Notably, we observed increased protein expression and localization of EMAPII in cells within the distal micro vessels that expressed activated pSPHK2 in human PH lung sections suggesting a potential interaction between EMAPII and SPHK2 in a PH setting.

This study is a sentinel step in the PH research field where epigenetic equilibrium is disrupted in part due to increased H3K9 acetylation in human Group 1 PH lung tissues. With the discovery of EMAPII altered acetylome in vascular SMCs, the potential targets that could modulate hyperproliferation of SMCs were tempting. Mechanistic investigations determined that the ectopic expression of OCT4, SOX2 and KLF4 can initiate the reprogramming of somatic cells to induce pluripotent stem cells (iPSCs) that closely resemble embryonic stem cells through epigenetic remodeling mechanisms. rEMAPII treatment induced a significant expression of SOX2 and KLF4 in vascular SMCs (See FIG. 14F,14G). Importantly, only enhancers of KLF4 were under local hyperacetylation of histone H3K9 in CUT&RUN studies. Numerous studies emphasize the role of KLF4 in vascular SMC proliferation, phenotypic switching of PASMC from contractile to synthetic and KLF4 involvement in vascular gene transcription thorough histone acetylation. This study establishes a novel role for the EMAPII/SPHK2/S1P/histone H3K9 acetylation axis in the progression of PH through reprogramming histone acetylome in vascular SMCs.

1. A method of reducing pulmonary artery vascular remodeling in a patient diagnosed with pulmonary arterial hypertension, said method comprising
administering an inhibitor of sphingosine kinase 2 to said patient.

2. The method of claim 1 wherein the inhibitor is an interfering RNA that targets sphingosine kinase 2 (SPHK2).

3. The method of claim 2 wherein the interfering RNA is complementary to a nucleic acid sequence common to each of the four splice variants of SPHK2 (SPHK2-a,b,c,d).

4. The method of claim 1 wherein the interfering RNA is selected from the group consisting of CAAGGCAGCUCUACACUCA (SEQ ID NO: 1) GAGACGGGCUGCUCCAUGA (SEQ ID NO: 2), GCUCCUCCAUGGCGAGUUU (SEQ ID NO: 3), CCACUGCCCUCACCUGUCU (SEQ ID NO: 4), and complements thereof.

5. The method of claim 1 wherein the inhibitor comprises a compound of the general structure: 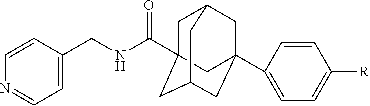
where R is halo.

6. A method of treating pulmonary arterial hypertension, said method comprising decreasing histone 3 acetylation at the lysine at position 9 (H3K9 acetylation) in the cells of a patient diagnosed with pulmonary arterial hypertension.

7. The method of claim 6 wherein H3K9 acetylation is decreased by administration of a pharmaceutical composition comprising an agent that
i) decreases sphingosine kinase 2 activity; or
ii) decreases Kruppel-like factor (KLF4) activity; or
iii) a combination of i) and ii).

8. The method of claim 7 wherein said method comprises
administering an inhibitor of sphingosine kinase 2 to said patient.

9. The method of claim 8 wherein the inhibitor is an interfering RNA that targets sphingosine kinase 2.

10. The method of claim 9 wherein the interfering RNA is selected from the group consisting of CAAGGCAGCUCUACACUCA (SEQ ID NO: 1) GAGACGGGCUGCUCCAUGA (SEQ ID NO: 2), GCUCCUCCAUGGCGAGUUU (SEQ ID NO: 3), CCACUGCCCUCACCUGUCU (SEQ ID NO: 4), and complements thereof.

11. The method of claim 8 wherein the inhibitor comprises a compound having the general structure: 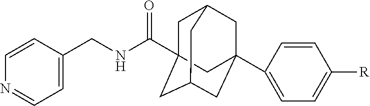
where R is halo.

12. The method of claim 8 wherein the inhibitor is a small molecule or antibody that targets sphingosine kinase 2.

13. The method of claim 6, wherein the method of decreasing the acetylation of the lysine at position 9 of histone H3 comprises reducing a rate of the thickening of distal pulmonary artery wall in the patient.

14. The method of claim 6, wherein the method of decreasing the acetylation of the lysine at position 9 of histone H3 comprises preventing or reducing remodeling of smooth muscle (SMC) and endothelial (EC) cells in the patient.

15. The method of claim 1 wherein the inhibitor is a small molecule or antibody that targets sphingosine kinase 2 (SPHK2).

16. The method of claim 1, wherein the method of reducing pulmonary artery vascular remodeling in a patient diagnosed with pulmonary arterial hypertension comprises reducing a rate of the thickening of distal pulmonary artery wall in the patient.

17. The method of claim 1, wherein the method of reducing pulmonary artery vascular remodeling in a patient diagnosed with pulmonary arterial hypertension comprises preventing or reducing remodeling of smooth muscle (SMC) and endothelial (EC) cells in the patient.
